(19) United States
(12) Patent Application Publication
(10) Pub. No.: US 2006/0194821 A1
(43) Pub. Date: Aug. 31, 2006

(54) Compounds inhibiting the aggregation of superoxide dismutase-1

(75) Inventors: Peter Lansbury, Brookline, MA (US); Andrew Choi, Dorchester, MA (US); Soumya Ray, Cambridge, MA (US); Richard Nowak, Philadelphia, PA (US)

Correspondence Address: FITCH, EVEN, TABIN & FLANNERY, P. O. BOX 65973, WASHINGTON, DC, 20035, US

(73) Assignee: The Brigham and women's Hospital, Inc., Boston, MA (US)

(21) Appl. No.: 11/354,896

(22) Filed: Feb. 16, 2006

Related U.S. Application Data
(60) Provisional application No. 60/653,983, filed on Feb. 18, 2005.

(51) Int. Cl. A61K 31/52; A61K 31/165

(52) U.S. Cl. 514/263.23; 514/617

(57) ABSTRACT

The invention is directed to methods of inhibiting the rate at which superoxide dismutse-1 (SOD) aggregates using compounds that stabilize SOD dimers. The methods are useful in the study and therapy of amyotrophic lateral sclerosis. The invention also includes assays that can be used to identify compounds that stabilize dimers and SOD molecules that have been modified for use in these assays.

### CROSS REFERENCE TO RELATED APPLICATIONS

The present application claims priority to and the benefit of U.S. provisional patent application 60/653,983, filed Feb. 18, 2005, which is incorporated herein by reference.

### STATEMENT OF GOVERNMENT SUPPORT

This invention was made with United States government support awarded by the Department of Health and Human Services as NIH Grant No.: AG08470. The United States may have certain rights in the invention.

### FIELD OF THE INVENTION

The present invention is directed to compounds that inhibit the aggregation of superoxide dismutase (SOD). Since aggregation of this protein has been associated with the development of amyotrophic lateral sclerosis (ALS) and other neurological diseases,” the inventive compounds are useful as therapeutic agents in treating and preventing ALS. They may also be used as tools by scientists studying the pathogenesis of ALS and other related diseases. In addition, the invention provides screening assays useful in identifying compounds that stabilize SOD and dimers thereof and/or prevent the aggregation of SOD. The invention also provides modified forms of SOD that are particularly useful in the inventive assays.

### BACKGROUND OF THE INVENTION

Amyotrophic lateral sclerosis (ALS), or Lou Gehrig's disease, is a fatal motor neuron disease that affects over 35,000 Americans and many more individuals world-wide. Approximately 20% of ALS cases are monogenic and autosomal dominant (familial ALS, FALS). The most common cause of FALS is point mutations in the gene encoding superoxide dismutase-1 (SOD-1), a β-sheet-rich dimeric metalloenzyme that is normally responsible for scavenging superoxide ion (1, 2).

SOD-1 is an abundant, ubiquitously expressed protein long-known to be involved in oxidative chemistry. However, its dismutase activity does not appear to be associated with ALS pathogenesis. For example, expression of ALS-linked SOD-1 mutant protein in rodents provokes progressive motor neuron disease independent of the protein dismutase activity (Bruijn et al. Science 281:1851-1854 (1998); incorporated herein by reference), and the deletion of the SOD-1 gene does not cause motor neuron disease in mice (Reaume et al. Nat. Genet. 13:43-47 (1996); incorporated herein by reference).

Studies with transgenic mice suggest that FALS may result from a “gain of toxic function” due to aggregation of the mutant form of SOD-1 (3). Since the 114 known SOD-1 FALS mutations are distributed throughout the primary sequence and tertiary structure of the SOD-1 protein, it is proposed that the mutations affect, in various ways, the structural stability of SOD-1 (1, 2). For example, one or more FALS SOD-1 mutations have been linked to decreased metal binding (4-6), decreased formation of a stabilizing intramolecular disulfide (7), decreased structural stability, and increased propensity to monomerize (8) and aggregate (7, 9-14). Occupancy of the zinc and copper binding sites (one each per subunit) may prevent SOD-1 aggregation (10). Thus the prevention of SOD-1 demetallation could slow the onset and progression of FALS, but a practical means for doing so in vivo has been elusive.

### SUMMARY OF THE INVENTION

A strategy for treating and/or preventing ALS and other related diseases is based upon the observation that SOD-1 is normally found in the form of homodimers and that the dissociation of these dimers may occur prior to aggregation of SOD. In fact, there is evidence suggesting that mutations in SOD destabilize the dimers and thereby subsequently lead to SOD aggregation (16, 10). Any compound that can stabilize the SOD-1 dimer and thereby prevent the aggregation of SOD-1 is useful in the present invention. In certain embodiments, the compound binds at the interface between the two subunits of the SOD-1 homodimer. The importance of this binding site, which includes amino acids Gly56, ThrA54, AsnA53, LysA9, CysA146, ValA148, ValA7, GlyB51, Thr116, and Gly147, has been validated by mutagenesis.

The present invention stems from an in silico screening program to find drug-like molecules (e.g, small molecules, peptides, proteins, drug-like molecules, etc.) that stabilize the SOD-1 dimer. Approximately 1.5 million molecules from commercial databases were docked at the dimer interface. Of the 100 molecules with the highest predicted binding affinity, fifteen significantly inhibited in vitro aggregation and denaturation of A4V, a FALS-linked variant of SOD-1. In the presence of several of these molecules, A4V and other FALS-linked SOD-1 mutants such as G93A and G85R behaved similarly to wild-type SOD-1, suggesting that these compounds should be effective therapeutics against FALS. These compounds are known in the art and can be synthesized using standard, known, methods. By examining the structures of the compounds, another group of related compounds was identified which were also found to be active at inhibiting SOD aggregation. These compounds will be of value to scientists studying ALS and in the treatment of this disease and related diseases.

In one aspect, the invention is directed to a method of inhibiting the aggregation of superoxide dismutase (e.g., SOD-1) by contacting superoxide dismutase in vitro or in vivo with an effective amount of a compound of formula I: 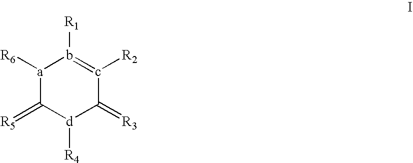
where:
a, b, c and d are each independently selected from the group consisting of: C, and N; R₁, R₂ and R₆ are each independently hydrogen, halogen, cyano, hydroxyl, amino, C₁-C₆ aliphatic (e.g., C₁-C₆ alkyl), or —(CH₂)ₙ-Z-CH₂)ₘ—R₇; where Z is —O—, —S—, —CR₁₂— or —NR′—, wherein R′ is hydrogen, halogen, or C₁-C₆ aliphatic (e.g., C₁-C₆ alkyl such as a methyl);
R₇ is H, CH₃, acyl, or a aryl or heteroaryl moiety optionally substituted at one or more positions with a halogen (e.g., F, Cl, Br, or I), a (C₁-C₆) aliphatic, a carbocyclic or heterocyclic moiety, —OH or —NH₂;
n is an integer from 0-3 inclusive;
m is an integer from 0-3 inclusive;
and where one or more single bonds in —CH₂)ₙ-Z-CH₂)ₘ may be replaced with a double bond;
when R₁ is —CH₂)ₙ-Z-(CH₂)ₘ—R₇, R₂ and R₆ may also each independently be selected from: H, a halogen, a (C₁-C₆) aliphatic, —OH or —NH₂;
when R₂ is —(CH₂)ₙ-Z-(CH₂)ₘ—R₇, R₁ and R₂ may also each independently be selected from: H, a halogen, a (C₁-C₆) aliphatic, —OH or —NH₂;
when R₆ is —(CH₂)ₙ-Z-(CH₂)ₘ—R₇, R₁ and R₂ may also each independently be selected from: H, a halogen, a (C₁-C₆) aliphatic, —OH or —NH₂;
R₃ and R₅ are each independently either O or S; and
R₄ is H, a halogen, a (C₁-C₆) aliphatic (e.g., methyl), —OH or —NH₂.

In certain embodiments:

a) a is N; or

b) c is N; or

c) d is N; or

d) a is C; or

e) c is C; or

f) d is C.

In certain embodiments:

a) at least one of R₁, R₂, and R₆ is hydrogen; or

b) at least two of R₁, R₂, and R₆ are hydrogen; or

c) at least one of R₁, R₂, and R₆ is methyl.

In certain embodiments:

a) Z is S; or

b) Z is O; or

c) Z is —NH—; or

d) Z is —NMe—; or

e) Z is —CH₂—.

In certain embodiments:

a) R₄ is hydrogen; or

b) R₄ is methyl.

In certain embodiments:

a) R₇ is hydrogen; or

b) R₇ is acyl; or

c) R₇ is acetyl; or

d) R₇ is —CO—R₇′, wherein R₇′ is C₁-C₆ alkyl (e.g., methyl, ethyl, propyl); alkoxy (e.g., methoxy, ethoxy); hydroxy; amino; substituted or unsubstituted aryl; substituted or unsubstituted heteroaryl; substituted or unsubstituted non-aromatic carbocyclic; or substituted or unsubstituted non-aromatic heterocyclic. In certain embodiments, R₇′ is a moncylic or bicyclic aryl or heteroaryl moiety optionally subsituted as described above. In certain embodiments, R₇′ is a monocyclic five- or six-membered aryl or heteroaryl moiety optionally substituted as described above. In yet other embodiments, R₇′ is an optionally substituted phenyl moiety. In certain embodiments, R₇′ is unsubstituted phenyl. In particular embodiments, R₇′ is a monosubstituted phenyl moiety. In other embodiments, R₇′ is a disubstituted phenyl moiety. In yet other embodiments, R₇′ is a trisubstituted phenyl moiety. In certain embodiments, R₇′ is an ortho-substituted phenyl moiety. In other embodiments, R₇′ is a meta-substituted phenyl moiety. In yet other embodiments, R₇′ is a para-substituted phenyl moiety (e.g. para-methylphenyl). In certain embodiments, R₇′ is a halogen-substituted phenyl moiety (e.g., ortho-halophenyl; ortho-chlorophenyl; ortho-fluorophenyl; para-halophenyl; para-chlorophenyl; para-fluorophenyl;). In certain embodiments, R₇′ is non-aromatic carbocyclic. In other embodiments, R₇′ is non-aromatic heterocyclic.

In other embodiments:

a) R₇ is —CH₃; or

b) R₇ is ethyl; or

c) R₇ is propyl; or

d) R₇ is butyl.

In certain embodiments R₇ is a moncylic or bicyclic aryl or heteroaryl moiety optionally subsituted as described above. In certain embodiments:
a) R₇ is a monocyclic five- or six-membered aryl or heteroaryl moiety optionally substituted as described above; or
b) R₇ is an optionally substituted phenyl moiety; or
c) R₇ is unsubstituted phenyl.
   In embodiments where R₇ is a phenyl moiety, it may be: monosubstituted; disubstituted; or a trisubstituted. In certain embodiments: R₇ is an ortho-substituted phenyl moiety; or R₇ is a meta-substituted phenyl moiety; or R₇ is a para-substituted phenyl moiety (e.g. para-methylphenyl). In certain embodiments, R₇ is a halogen-substituted phenyl moiety (e.g., ortho-halophenyl; ortho-chlorophenyl; ortho-fluorophenyl; para-halophenyl; para-chlorophenyl; para-fluorophenyl). In certain embodiments, R₇ is non-aromatic carbocyclic or a non-aromatic heterocyclic.

In certain embodiments:

a) R₇ is C₁-C₆ alkyl; or

b) R₇ is C₁-C₆ alkenyl; or

c) R₇ is C₁-C₆ alkynyl; or

d) R₇ is C₁-C₃ alkyl; or

e) R₇ is C₁-C₃ alkenyl; or

f) R₇ is C₁-C₃ alkynyl.

In certain embodiments, m is 0 or 1. In certain embodiments, n is 0 or 1. In certain embodiments, n is 0. In certain embodiments, n is 1. In certain embodiments, n is 2. In certain embodiments, m is 0. In certain embodiments, m is 1. In certain embodiments, m is 2. In certain embodiments, one of m or n is 0, and the other is 1. In certain embodiments, n is 0 and m is 1. In certain embodiments, both m and n are 0.

In certain embodiments, both R₃ and R₅ are O. In other embodiments, both R₃ and R₅ are S. In yet other embodiments, one of R₃ and R₅ is O, and the other is S.

In preferred embodiments a, b, c and d are each independently selected from the group consisting of C and N with the proviso that a, b, c and d are not all N or, alternatively, that no more than two of a, b, c and d may be N or, alternatively, that no more than one of a, b, c and d may be N. It is also preferable that only one of R₁, R₂ or R₆ be —(CH₂)ₙ-Z-(CH₂)ₘ—R₇, with the other positions being hydrogen; halogen; cyano; a (C₁-C₆)alkyl (e.g., methyl); OH; or NH₂.

In certain embodiments, the compound of formula I is of the formula (Ia): 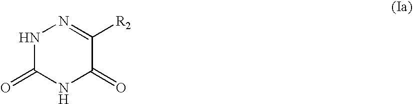
wherein R₂ is defined as above.

In certain embodiments, the compound of formula I is of the formula (Ib): 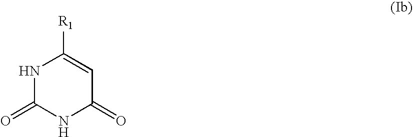
wherein R₁ is defined as above.

In certain embodiments, the compound of formula I is of the formula (Ic): 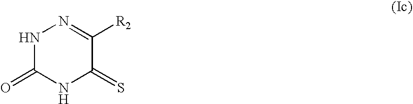
wherein R₂ is defined as above.

In certain embodiments, the compound of formula I is of the formula (Id): 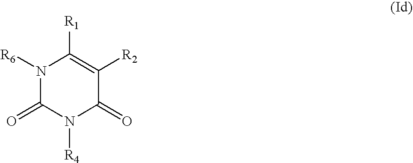
wherein R₁, R₂, R₄, and R₆ are defined as above.

In certain embodiments, the compound of formula I is of the formula (Ie): 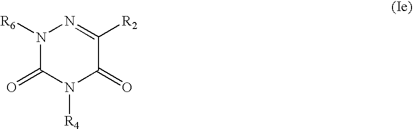
wherein R₂, R₄, and R₆ are defined as above.

In certain embodiments, the compound of formula I is of the formula (If): 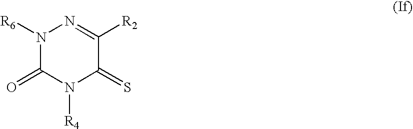
wherein R₂, R₄, and R₆ are defined as above.

In certain embodiments, the compound of formula I is of the formula (Ig): 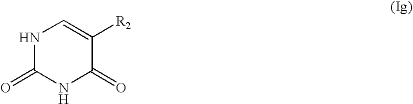
wherein R₂ is defined as above.

In certain embodiments, the compound of formula I is of the formula (Ii): 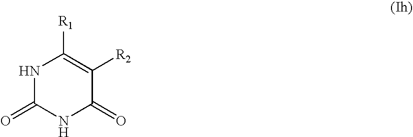
wherein R₁ and R₂ are defined as above.

In certain embodiments, the compound of formula I is of the formula (Ii): 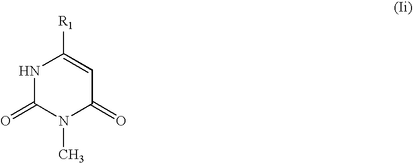
wherein R₁, R₂, R₄, or R₆ is defined as above.

In another aspect, the invention is directed to a method of inhibiting the aggregation of superoxide dismutase or treating a subject using compounds of formula II: 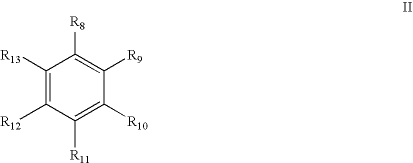
wherein:
R₈, R₉ and R₁₃ are each independently selected from:
group a): 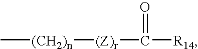
where n is an integer from 0 to 3 inclusive; r is an integer from 1-3 inclusive; Z is: —S—, —CR′₂— or —NR′—, wherein R′ is hydrogen, halogen, or C₁-C₆ aliphatic (e.g., methyl); and R₁₄ is a (C₁-C₆) aliphatic, a halogen, OH, or —NH—R₁₅; where R₁₅ is H, NH₂, OH, or a (C₁-C₃) aliphatic;
group b): 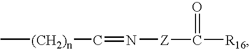
where n is an integer from 0 to 3 inclusive; Z is: —S—, —CR′₂— or —NR′—, wherein R′ is hydrogen, halogen, or C₁-C₆ aliphatic (e.g., methyl); and R₁₆ is selected from: —H, —OH, a halogen NH₂, a (C₁-C₃) aliphatic, and a phenyl optionally substituted at one or more positions with a halogen, —OH, a (C₁-C₃) aliphatic, or —NH₂;
group c): 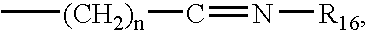
wherein n is an integer from 0 to 3, inclusive; preferably, n is 0 or 1; and R₁₆ is selected from: hydrogen, —OH, a halogen, —NH₂, a (C₁-C₆) aliphatic, and a phenyl optionally substituted at one or more positions with a halogen (e.g., para-fluorophenyl, para-chlorophenyl, etc.), —OH, a (C₁-C₃) aliphatic, or —NH₂;
group d): —(CH₂)ₙ-phenyl, where n is an integer from 0 to 3 inclusive, and the phenyl may optionally be substituted at one or more positions with a halogen, —OH, a (C₁-C₃) aliphatic, alkoxy, or —NH₂; and
when R₈ is any of group a), group b), group c), or group d), R₉ and R₁₃ may also each be independently selected from: H, a halogen, acyl, a (C₁-C₃) aliphatic, —OH, alkoxy, and —NH₂;
when R₉ is any of group a), group b), group c), or group d), R₈ and R₁₃ may also each be independently selected from: H, a halogen, acyl, a (C₁-C₃) aliphatic, —OH, alkoxy, and —NH₂;
when R₁₃ is any of group a), group b), group c), or group d), R₈ and R₉ may also each be independently selected from: H, a halogen, acyl, a (C₁-C₃) aliphatic, —OH, alkoxy, and —NH₂.
R₁₀, R₁₁, and R₁₂ are each independently selected from the group consisting of H, a halogen, acyl, a C₁-C₆ aliphatic (e.g., a C₁-C₆ alkyl), —OH, alkoxy, and —NH₂, and any one of R₁₀, R₁₁, and R₁₂ may also be —(CH₂)ₙ-phenyl, wherein n is an integer from 0 to 3 inclusive and the phenyl may optionally be substituted at one or more positions with a halogen, acyl, a C₁-C₆ aliphatic (e.g., a C₁-C₆ alkyl), —OH, alkoxy, or —NH₂. Preferably, only one of variables R₈, R₉ and R₁₃ corresponds to group a), b) or c); with the other two variables being H, a halogen, acyl, a C₁-C₆ aliphatic (e.g. a C₁-C₆ alkyl)-OH, alkoxy, and —NH₂.

In certain embodiments, all of R₁₀, R₁₁, and R₁₂ are hydrogen. In other embodiments, at least two of R₁₀, R₁₁, and R₁₂ are hydrogen. In still other embodiments, at least one of R₁₀, R₁₁, and R₁₂ is hydrogen.

In certain embodiments, at least one of R₁₀, R₁₁, and R₁₂ are halogen. In certain other embodiments, at least one of R₁₀, R₁₁, and R₁₂ is bromine. In certain other embodiments, at least one of R₁₀, R₁₁, and R₁₂ is fluorine. In certain other embodiments, at least one of R₁₀, R₁₁, and R₁₂ is chlorine. In certain other embodiments, at least one of R₁₀, R₁₁, and R₁₂ is —OH. In certain other embodiments, at least one of R₁₀, R₁₁, and R₁₂ is methyl. In certain other embodiments, at least one of R₁₀, R₁₁, and R₁₂ are —NH₂.

The invention also includes methods of inhibiting the aggregation of superoxide dismutase or treating a subject using compounds of formula III: 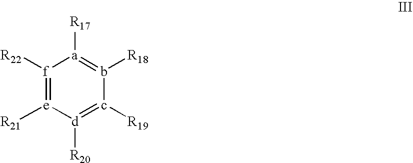
wherein:
a, b, c, d, e, and f are each independently C or N;
at least one of R₁₇, R₁₈ and R₂₂ is 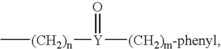
where Y is C or S, n and m are each independently an integer from 0 to 3 inclusive, and the phenyl may optionally be substituted at one or more positions with a halogen, a (C₁-C₃)alkyl, OH or NH₂;
in cases where R₁₇, R₁₈ or R₂₂ is not 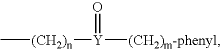
it is independently selected from H, a halogen, a (C₁-C₃)alkyl, OH and NH₂;
at least one of R₁₉, R₂₀ and R₂₁ is 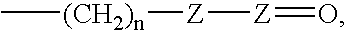
wherein Z is C, N or S,
n is an integer from 0 to 3;
in cases where R₁₉, R₂₀ or R₂₁ is not 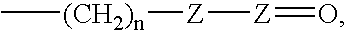
it is independently selected from H, a halogen, a (C₁-C₃)alkyl, OH and NH₂.

In certain embodiments, only one of a, b, c, d, e, or f is N. In other embodiments, only two of a, b, c, d, e, or f is N.

In preferred embodiments, no more than three of a, b, c, d, e and f are N; no more than one of R₁₇, R₁₈ and R₂₂ is 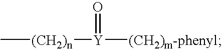
and no more than one of R₁₉, R₂₀ and R₂₁ is 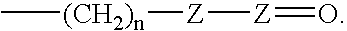
In other embodiments the number of nitrogens in formula II is limited to two or to one.

In certain embodiments, the compound of formula III is of the formula (IIIa): 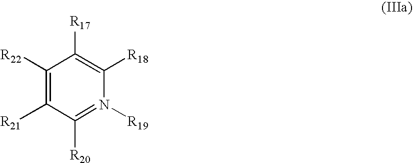
wherein R₂ is defined as above.

In another aspect, the invention is directed to methods of inhibiting the aggregation of superoxide dismutase or treating a subject which utilize compounds of formula IV: 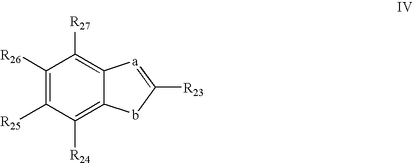
wherein a and b are S, O, —CR′— R′ is hydrogen, halogen, or C₁-C₆ aliphatic (e.g., C₁-C₆ alkyl such as a methyl), N or NR₂₃ or S;
R₂₄, R₂₅, R₂₆ and R₂₇ are selected from H, a halogen, a (C₁-C₃)alkyl, OH, and NH₂; and, at each occurrence of R₂₃ is independently substituted or unsubstituted, branched or unbranched aliphatic or heteroalipatic; substituted or unsubstituted aryl or heteroaryl; 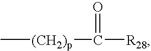
where p is an integer from 0 to 6 inclusive, hydrogen or C₁-C₆ aliphatic; and R₂₈ is selected from a (C₁-C₆) aliphatic (e.g., a C₁-C₆ alkyl), (C₁-C₆)alkoxy, OH, a halogen, —NHR′, —NR′₂, NH₂, NH—NH₂, and NH—CH₃, wherein R′ is halogen, OH, ureido, substituted or unsubstituted, branched or unbranched aliphatic or heteroaliphatic, substituted or unsubstituted aryl or heteroaryl, or acyl. In certain embodiments, R₂₈ is either NH₂ or NH—NH₂. In other embodiments, R₂₈ is —NHR′, wherein R′ is an optionally a substituted aryl (e.g., phenyl) or heteroaryl moiety (e.g., imidazolyl, thiazolyl, oxazolyl, pyridinyl, etc.). In certain embodiments, R₂₈ is —NR′₂, wherein at least one of R′ is methyl. In certain embodiments, R₂₃ is C₁-C₃ alkyl (e.g., methyl).
In certain embodiments, all of R₂₄, R₂₅, R₂₆, and R₂₇ are hydrogen. In certain embodiments, a is N. In other embodiments, b is S.

In certain embodiments, the compound of formula IV is of the formula (IVa): 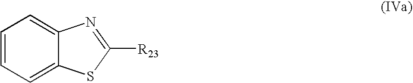
wherein R₂₃ is defined as above.
In certain embodiments, the compound of formula IV is of the formula (IVb): 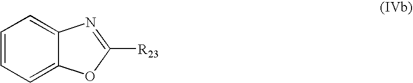
wherein R₂₃ is defined as above.

In certain embodiments, the compound of formula IV is of the formula (IVc): 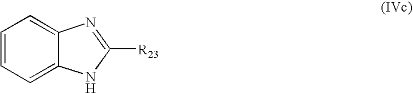
wherein R₂₃ is defined as above.

In certain embodiments, the compound of formula IV is of the formula (IVd): 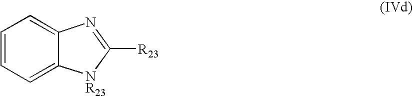
wherein R₂₃ is defined as above.

In certain embodiments, the compound of formula IV is of the formula (IVe): 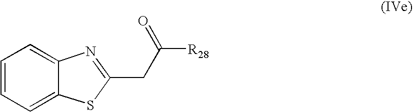
wherein R₂₈ is defined as above.

In certain embodiments, the compound of formula IV is of the formula (IVf): 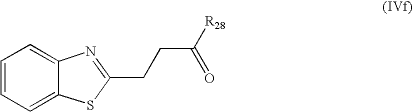
wherein R₂₈ is defined as above.

In certain embodiments, the compound of formula IV is of the formula (IVg): 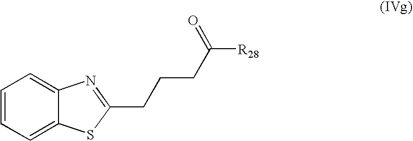
wherein R₂₈ is defined as above.

Other compounds that may be used to inhibit the aggregation of SOD or to treat a subject have the structure of formula V: 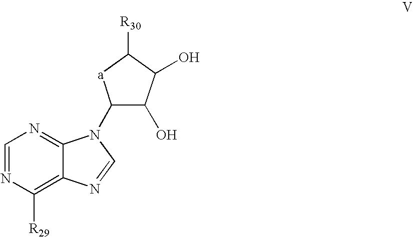
wherein:
a is selected from —O—, —CH₂—, —NH—, or —S—;
R₂₉ is selected from a halogen, OH, (C₁-C₆) aliphatic, and NH₂; and
R₃₀ is —(CH₂)_{q}OH or —(CH₂)_{q}—OP(O)(OH)₂, wherein q is an integer from 1 to 3 inclusive, preferably, q is 1.
In certain embodiments, a is O whereas in others it is not O. In other embodiments, a is —CH₂—. In yet other embodiments, a is S. In still other embodiments, a is —NH—.
In certain embodiments, R₂₉ is —NH₂. In certain embodiments, R₂₉ is methyl.

The compounds useful in the inventive methods may also have the structure of formula VI:

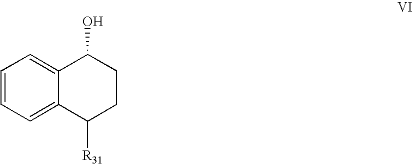

wherein one or more positions in the ring structure of formula VI may be substituted with a group selected from a halogen, a C₁-C₆ aliphatic (e.g., C₁-C₆ alkyl; OH; and NH₂; and
R₃₁ is 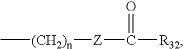
where n is an integer from 0 to 3 inclusive (preferably, 1 or 2); Z is —CH₂—, —NH—, —O—, or —S-(preferably, —NH—); and R₃₂ is selected from a halogen, —OH, C₁-C₆ aliphatic (e.g., a C₁-C₆ alkyl), and —NH₂ (preferably, —NH₂).
In certain embodiments, R₃₁ is —NH—CO—NH₂.

In another aspect, the invention is directed to a method of inhibiting the aggregation of superoxide dismutase or treating a subject using a compound of formula VII: 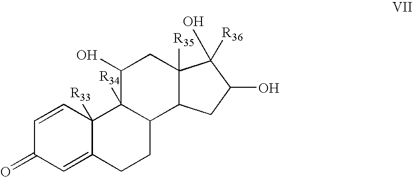
wherein:
R₃₃, R₃₄ and R₃₅ are each independently selected from the group consisting of hydrogen, halogen, (C₁-C₆) aliphatic, —OH, and —NH₂; and
R₃₆ is 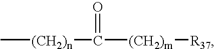
where n and m are each independently an integer from 0 to 3 inclusive and R₃₇ is selected from the group consisting of hydrogen, halogen, —CH₃, —OH; and —NH₂.
In certain embodiments, R₃₃ is C₁-C₃ alkyl. In certain particular embodiments, R₃₃ is methyl. In certain embodiments, R₃₄ is hydrogen. In other embodiments, R₃₄ is halogen. In yet other embodiments, R₃₄ is fluorine. In certain embodiments, R₃₅₃ is C₁-C₃ alkyl. In certain particular embodiments, R₃₅ is methyl. In certain embodiments, R₃₆ is

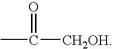

In an especially preferred aspect, the invention is directed to a method of inhibiting the aggregation of superoxide dismutase or treating a subject using a compound of formula VIII: 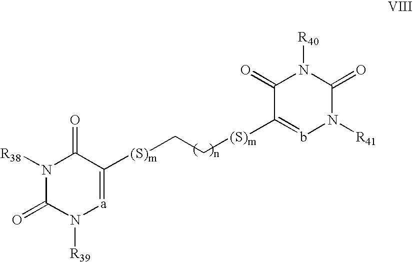
wherein:
R₃₈, R₃₉, R₄₀ and R₄₁ is each independently selected from the group consisting of H, C₁-C₆ aliphatic, aryl, heteroaliphatic, heteroaryl, aryl alkyl, and heteroarylalkyl;
a and b are each independently —CH— or —N—;
n is an integer from 0-6 inclusive; and
m is an integer from 0-1 inclusive.
In certain embodiments, R₃₈ is hydrogen, in others R₃₉ is hydrogen; in others, R₄₀ is hydrogen and in others R₄₁ is hydrogen. In certain embodiments, m is 1, in others, n is at least 1, in others n is 1, in others both a and b are —CH— and in others, both a and b are —N—.

In another aspect, the invention is directed to a method of inhibiting the aggregation of superoxide dismutase or treating a subject using a compound of formula IX: 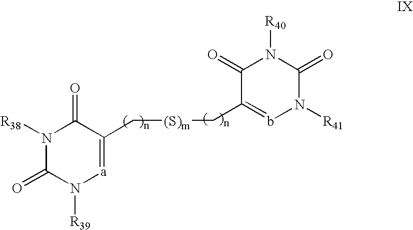
wherein:
R₃₈, R₃₉, R₄₀ and R₄₁ is each independently selected from the group consisting of H, C₁-C₆ aliphatic, aryl, heteroaliphatic, heteroaryl, arylalkyl, and heteroarylalkyl;
a and b are each independently —CH— or —N—;
n is an integer from 0-6 inclusive; and
m is an integer from 0-1 inclusive.
In certain embodiments, R₃₈ is hydrogen, in others R₃₉ is hydrogen, in others R₄₀ is hydrogen, in others R₄₁ is hydrogen, in others m is 1, in others n is at least 1, in others n is 1, in others both a and b are —CH— and in others both a and b are —N—.

In an especially preferred aspect, the invention is directed to a method of inhibiting the aggregation of superoxide dismutase or treating a subject using a compound of formula X: 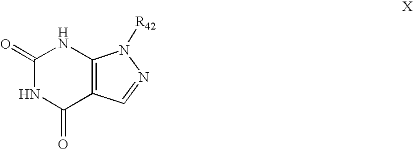
wherein:
R₄₂ is selected from the group consisting of H, C₁-C₆ aliphatic, aryl, heteroaliphatic, heteroaryl, arylalkyl, and heteroarylalkyl.
In certain embodiments, R₄₂ is aryl, in others R₄₂ is heteroaryl, in others R₄₂ is an unsubstituted phenyl moiety and in others R₄₂ is a substituted phenyl moiety.

Specific compounds for use in the inventive methods described above include:
a) N-nitroso-5-(phenylsulfinyl)pyridin-2-amine;
b) 6-[(4-chlorophenyl)amino]pyrimidine-2,4(1H,4H)-dione;
c) 6-[(4-chlorobenzyl)thio]1,2,4-triazine-3,5(2H,4H)-dione;
d) 4-bromo-2-{(E)-[(4-fluorophenyl)imino]methyl}phenol;
e) 6-(ethylthio)-thioxo-4,5-dihydro-1,2,4-triazin-3(2H)-one;
f) 2-[2-(2-amino-4-methylphenyl)ethyl]-5-methylaniline;
g) 6-[(4-fluorobenzyl)thio]-1,2,4-triazine-3,5(2H,4H)-dione;
h) 2-(3-fluorophenyl)hydrazinecarboxamide;
i) 3-benzyl-2-hydroxylbenzohydrazide;
j) 4-hydroxybenzaldehyde semicarbazone;
k) 4-(1,3-benzothiazol-2-yl)butanamide;
l) 2-(1H-benzimidazol-2-yl)acetohydrazide;
m) N-[(1R,4R)-4-hydroxy-1,2,3,4-tetrahydronaphthalen-1-yl]urea;
n) trimacicolone;
o) 6-amino-methy-adenosine;
p) methyl3-(3,5-dioxo-2,3,4,5-tetrahydro-1,2,4-triazin-6-yl)propanoate;
q) 6-[(3,5-dimethyl-1H-pyrazol-4-yl)thio]-1,2,4-triazine-3,5(2H,4H)-dione;
r) 3-methyl-6-[methyl(phenyl)amino]pyrimidine-2,4(1H,3H)-dione;
s) 2-(2-methyl-1H-benzimidazol-1-yl)acetamide;
t) hydroxy(oxo) {4-[(2-oxo-1,2,3,6-tetrahydopyrimidin-4-yl)amino]phenyl}ammonium;
u) 6-[(2-chlorophenyl)amino]pyrimidine-2,4(1H,3H)-dione;
v) 6-[(4-pyrrolidin-1-ylphenyl)amino]pyrimidine-2,4(1H,3H)-dione;
w) 6-[(4-methylphenyl)amino]pyrimidine-2,4(1H,3H)-dione;
x) 5-ethylpyrimidine-2,4(1H,3H)-dione;
y) 6-anilino-2,4-dioxo-1,2,3,4-tetrahydropyrimidine-5carbonitrile;
z) 6-anilino-1-methylpyrimidine-2,4(1H,3H)-dione;
aa) 3-methyl-6-[(4-methylphenyl)amino]pyrimidine-2,4(1H,3H)-dione;
bb) 1-phenyl-1H-pyrazolo[3,4-d]pyrimidine-4,6(5H,7H)-dione;
cc) 6-[(4-chlorophenyl)amino]-3-methylpyrimidine-2,4(1H,3H)-dione;
dd) 6-(allylthio)-1,2,4-triazine-3,5(2H,4H)-dione;
ee) ethyl[(3,5-dioxo-2-tetrahydrofuran-2-yl-2,3,4,5-tetrahydro-1,2,4-triazin-6-yl)thio]acetate;
ff) 6-[(imidazo[1,2-a]pyridin-2-ylmethyl)thio]-1,2,4-triazine-3,5(2H,4H)-dione;
gg) 6-[(1-naphthylmethyl)thio]-1,2,4-triazine-3,5 (2H,4H)-dione;
hh) 6-[(2-morpholin-4-yl-2-oxoethyl)thio]-1,2,4-triazine-3,5(2H,4H)-dione;
ii) 6-{[2-(4-methoxyphenyl)-2-oxoethyl]thio}-1,2,4-triazine-3,5(2H,4H)-dione;
jj) 6-{[2-(2-chlorophenyl)-2-oxoethyl]thio}-1,2,4-triazine-3,5(2H,4H)-dione;
kk) 6-{[(2-phenyl-1,3-thiazol-4-yl)methyl]thio}-1,2,4-triazine-3,5(2H,4H)-dione;
ll) 6-[(2-chloro-6-flourobenzyl)thio]-1,2,4-triazine-3,5(2H,4H)-dione;
mm) 4-(1,3-benzothiazol-2-yl)-N-phenylbutanamide;
nn) N-(aminocarbonyl)-2-(1,3-benzothiazol-2-yl)acetamide;
oo) 4-(1,3-benzothiazol-2-yl)butanoic acid;
pp) 3-(1,3-benzothiazol-2-yl)-N-1,3-thiazol-2-ylpropanamide;
qq) 3-(1,3-benzothiazol-2-yl-NN-dimethylpropanamide;
rr) methyl-4-(1,3-benzothiazol-2-yl)butanoate;
ss) 3-(1,3-benzothiazol-2-yl-N-phenylpropanamide;
tt) 3-(1,3-benzothiazol-2-yl)-N-methyl-N-phenylpropanamide;
uu) 2-[4-(1,3-benzothiazol-2-yl)piperidin-1-yl]acetamide;
vv) 6-(1-naphthylamino)pyrimidine-2,4(1H,3H)-dione;
ww) methyl-4-[(2,6-dioxo-1,2,3,6-tetrahydropyrimidin-4-yl)amino]benzoate;
xx) 6-{[(2,4-dioxo-1,2,3,4-tetrahydropyrimidin-5-yl)methyl]thio}-1,2,4-triazine-3,5(2H,4H)-dione;
yy) 1,2-di-[6-Mercapto-2H-[1,2,4]triazine-3,5-dione]ethane; and
zz) di-{5-[1H-pyrimidine-2,4-dione]methyl}thioether.

Of these, the particularly useful compounds are:
6-[(4-chlorophenyl)amino]pyrimidine-2,4(1H,4H)-dione;
6-[(4-chlorobenzyl)thio]1,2,4-triazine-3,5(2H,4H)-dione;
4-bromo-2-{(E)-[(4-fluorophenyl)imino]methyl}phenol;
6-[(4-fluorobenzyl)thio]-1,2,4-triazine-3,5(2H,4H)-dione; and
6-{[(2,4-dioxo-1,2,3,4-tetrahydropyrimidin-5-yl)methyl]thio}-1,2,4-triazine-3,5(2H,4H)-dione;
6-{[(2,4-dioxo-1,2,3,4-tetrahydropyrimidin-5-yl)methyl]thio}-1,2,4-triazine-3,5(2H,4H)-dione;
1,2-di-[6-Mercapto-2H-[1,2,4]triazine-3,5-dione]ethane; and
di-{5-[1H-pyrimidine-2,4-dione]methyl}thioether.

The ability to inhibit SOD aggregation is also of use to scientists studying ALS and related diseases and to clinicians working to develop effective treatments for these diseases. Thus, the compounds may be used in in vitro assays to assess their ability to stabilize SOD dimers or to prevent the aggregation of SOD dimers. The compounds described above may also be administered to test animals to study SOD dimer stabilization, the relationship between the rate at which SOD aggregates and the development of ALS signs and symptoms. They also may be given to patients for the purpose of slowing disease progression or preventing the disease. Since ALS is always severely debilitating and nearly always fatal, any drug that is not itself highly toxic and that preserves nerve function, even to a small degree, represents a significant advance.

It will be understood that the methods described herein may employ any pharmaceutically acceptable form of the compounds that are recognized in the art and particularly all pharmaceutically acceptable salts, derivatives, stereoisomers, isomers, tautomers, and pro-drugs. To the extent that the compounds were not previously known in the art or had no known function, the invention includes the compounds themselves. In particular the invention includes the compounds:
1,2-di-[6-Mercapto-2H-[1,2,4]triazine-3,5-dione]ethane; and
di-{5-[1H-pyrimidine-2,4-dione]methyl}thioether
   either alone or in any of the pharmaceutical compositions or dosage forms described herein.

The invention encompasses pharmaceutical compositions, particularly in unit dosage form, containing any of the compounds described above and methods of inhibiting the aggregation of superoxide dismutase using these pharmaceutical compositions, especially as a treatment for neurological diseases such as ALS. As used herein, the term “pharmaceutical composition” refers to a composition containing one or more of the compounds described above together with one or more pharmaceutically acceptable excipients. In the case of solid dosage forms, typical excipients would include pharmaceutically acceptable salts; buffering agents (e.g., phosphate or bicarbonate buffers); binders (e.g., polyvinyl pyrrolidone (PVP), hydroxypropyl cellulose (HPC), hydroxypropyl methyl cellulose (HPMC)); plasticizers (e.g., polysorbates; dimethyl phthalate, diethyl phthalate, triacetin, triethyl citrate, and polyethylene glycol (PEG)); lubricants (e.g., magnesium stearate); disintegrants (e.g., croscarmellose salts) etc. Flavoring agents, coloring agents and coatings may also be present. Liquid dosage forms, particularly those for parenteral administration, would include a sterile, pharmaceutically acceptable, aqueous or organic vehicle in which compounds are dissolved or suspended.

The term “unit dosage form” as used herein refers to a single entity for drug administration. For example, a single tablet, capsule or injection vial or ampoule would constitute a unit dosage form. Sufficient compound should be present to achieve a positive therapeutic effective when one or more unit doses are administered to a patient as measured using accepted clinical criteria. For example, in the case of ALS, a “therapeutically effective amount” would be an amount sufficient to slow the loss of motor function in a patient. This amount may be determined using methods well known in the art of pharmacology and, depending on the particular compound and dosage form used, could be anywhere from a few micrograms up to many milligrams. For example, a unit dosage form may have an amount of compound in the range of: 0.001-1000 mg; 0.01-500 mg; 0.01-50 mg; 0.1-50 mg etc. The invention also includes therapeutic packages in which the unit dosage forms are present in a labeled, finished pharmaceutical container, along with instructions on administering the dosage forms to a patient for the treatment of a disease such as ALS.

In another aspect, the invention is directed to an assay that can be used to screen for compounds that stabilize SOD dimers. In certain embodiments, this assay utilizes SOD molecules that have been labeled with fluorophores at specific sites that come into close proximity when dimers of SOD protein form. The fluorophores are chosen based on their ability to exchange energy when in close proximity. For example, when two pyrenes are brought close together as the result of dimerization, an excimer is formed that results in a shift in fluorescence absorbance. Similarly, fluorescence resonance energy transfer (FRET) assays may be performed by labeling one group of SOD molecules with an energy donor fluorophore and the other with an energy acceptor fluorophore. Homodimers (i.e., dimers in which both SOD molecules are the same and have only donor groups or only acceptor groups) are isolated and then incubated together. Upon dissociation and redimerization, mixed heterodimers (having one SOD that is unlabeled and one that is labeled) are formed and absorb at a characteristic wavelength. By carrying out incubations in both the presence and absence of a test compound, conclusions can be drawn as to whether the test compound stabilizes the dimer.

Sites in SOD that may be mutated to allow for the attachment of fluorophore include, for example, Gly51; Asp52; Thr54; Ala55; Ser59; Ala1; Thr2; Ala4; Val5; Val7; Lys9; Gly10; Asp11; Gly12; Gln15; Ser107; Gly108; Asp109; Cys111; Ile113; Gly114; Arg115; Thr116; and Leu117. In certain embodiments, the amino acid is changed to Cys, Ser, Lys, or other amino acids, depending on the particular fluorophore chosen for labeling. In certain embodiments, an SOD mutant in which the lysine at position 9 is changed to cysteine is used in the assay. The invention includes mutated forms of SOD protein, dimers of these mutated forms of SOD, dimers of fluorescently labeled SOD protein, and fluorescence assays used to determine the rate at which SOD dimers dissociate. The present invention also include polynucleotides encoding mutant SOD protein, vectors encoding mutant SOD proteins, and cells (e.g., bacterial cells (e.g., E. coli), yeast cells, insect cells, mammalian cells) transformed therewith.

### BRIEF DESCRIPTION OF THE DRAWINGS

FIG. 1: Chemical structures for 15 compounds found to inhibit A4V aggregation: The chemical name and structure for each of the compounds found to be effective in inhibiting aggregation are shown.

FIG. 2: Chemical structures for additional compounds: Based upon the results obtained with the 15 compounds shown in FIG. 1, the structural analogs shown in FIG. 2 were obtained and tested. All of these compounds were found to be active at inhibiting aggregation. Most of the compounds were purchased commercially. However two of the compounds, 1,2-di-[6-Mercapto-2H-[1,2,4]triazine-3,5-dione]ethane; and di-{5-[1H-pyrimi dine-2,4-dione]methyl}thioether were not available commercially and, instead, were synthesized using the procedure described in Example 3 and shown in FIG. 3.

FIG. 3: Chemical synthesis of compounds: FIG. 3 illustrates the method used for synthesizing 1,2-di-[6-Mercapto-2H-[1,2,4]triazine-3,5-dione]ethane; and di-{5-[1H-pyrimi dine-2,4-dione]methyl}thioether. The method is described in Example 3.

FIG. 4: Wild type SOD sequence. The naturally occurring amino acid (SEQ ID NO:1) and nucleotide (SEQ ID NO:2) sequences of SOD are shown in the figure.

FIG. 5: K9C SOD. The amino acid (SEQ ID NO:3) and nucleotide (SEQ ID NO:4) sequences of SOD mutated at amino acid 9 by replacing lysine with cysteine.

FIG. 6: A4V, K9C SOD: The amimo acid (SEQ ID NO:5) and nucleotide (SEQ ID NO:6) sequences of SOD that has been mutated at position 9 by replacing lysine with cysteine. This mutation facilitates fluorescent labeling. In addition, there is a second mutation at position 4 in which alanine has been replaced with valine.

FIG. 7: Models of cavity at SOD-1 dimer interface partially filled by mutagenesis: Panel (A) shows a surface representation of A4V mutant superoxide dismutase-1 dimer shaded to show the two subunits. A deep cavity at the dimer interface is highlighted by the box and indicates the drug binding site. The surface was generated using a water molecule as a probe. FIG. 7, panel B shows a close-up of the drug binding site with certain residues which form the pocket labeled—Val7, Gly147, and Val148 (each subunit contributes these three residues to the binding pocket.

FIG. 8: FIGS. 8A and 8B are different views of the drug binding pocket of SOD-1. Positively charged areas are shown in blue. Negatively charged areas are shown in red. Hydrophobic areas are shown in yellow and green. The residues making up the binding site include Gly56, Thr54, Asn53, Lys9, Cys146, Val148, Val7, Gly 51, Thr116, and Gly147. FIG. 8C shows the surface of the binding pocket.

FIG. 9: Agents docked in the SOD-1 binding pocket. 9A shows six different compounds docked in the binding site. FIG. 9B shows a collection of compounds docked in the binding pocket and also illustrates hydrophilic and hydrophobic regions of the binding pocket. FIG. 9C shows four different regions of the binding pocket and the distances between them. FIG. 9D includes exemplary cyclic structures which could occupy Site-1 and/or Site-2 in FIG. 9C. Linkers of the proper length are also included as 9E.

Definitions

Definitions of specific functional groups and chemical terms are described in more detail below. For purposes of this invention, the chemical elements are identified in accordance with the Periodic Table of the Elements. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties to the extent defined for individual structures and as accepted in the art. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein and unless otherwise indicated, the term “substituted” is contemplated to include all permissible substituents of organic compounds.

The term “acyl”, as used herein, refers to a carbonyl-containing functionality, e.g., —C(═O)R′, wherein R′ is an aliphatic, alycyclic, heteroaliphatic, heterocyclic, aryl, heteroaryl, (aliphatic)aryl, (heteroaliphatic)aryl, heteroaliphatic(aryl) or heteroaliphatic(heteroaryl) moiety.

The term “aliphatic”, as used herein, includes both saturated and unsaturated, straight chain (i.e., unbranched), branched, acyclic, cyclic, or polycyclic aliphatic hydrocarbons. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups.

The term “alkoxy” or “alkylthioxy” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexyl-amino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

Some examples of possible substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted to the extent indicated, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “heterocycloalkyl” or “heterocyclic”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

Specific heterocyclic and aromatic heterocyclic groups that may be included in the compounds of the invention include: 3-methyl-4-(3-methylphenyl)piperazine, 3 methyl-piperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piper-azine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl) piperazine, 4-(2-cyanophenyl)piperazine, 4-(2-ethoxyphenyl)piperazine, 4-(2-ethylphenyl) piperazine, 4-(2-fluorophenyl)piperazine, 4-(2-hydroxyethyl)piperazine, 4-(2-methoxyethyl) piperazine, 4-(2-methoxyphenyl)piperazine, 4-(2-methylphenyl)piperazine, 4-(2-methyl-thiophenyl) piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl) piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxy-phenyl)piperazine, 4-(3,4-dimethylphenyl)piperazine, 4-(3,4-methylenedioxyphenyl) piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethyl-ethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl) piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl) piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

Carbocycle”: The term “carbocycle”, as used herein, refers to an aromatic or non-aromatic ring in which each atom of the ring is a carbon atom.

“Hydrophobic”: The term “hydrophobic” refers to a moiety which tends not to dissolve in water and is fat-soluble. Hydrophobic moieties include, but are not limited to, hydrocarbons, such as alkanes, alkenes, alkynes, cycloalkanes, cycloalkenes, cycloalkynes, and aromatic compounds, such as aryls, certain saturated and unsaturated heterocycles and moieties that are substantially similar to the side chains of hydrophobic natural and unnatural alpha-amino acids, including valine, leucine, isoleucine, methionine, phenylalanine, alpha-aminobutyric acid, alloisoleucine, tyrosine, and tryptophan.

“Independently selected”: The term “independently selected” is used herein to indicate that the R groups can be identical or different.

“Ureido”: The term “ureido,” as used herein, refers to a urea group of the formula —NH—CO—NH₂.

“Effective amount”: The term “effective amount” means that a sufficient amount of compound is present to substantially reduce (e.g., by at least 10%, 20%, 30%, 40%, 50%, 60%, 70%, 80%, 90%, 95%, 98%, or 99%) the aggregation of SOD as compared to the aggregation in the absence of compound. In certain embodiments, it is the amount sufficient to stabilize SOD dimers. In other embodiments, the effective amount is the amount administered to a subject sufficient to prevent the signs or symptoms of ALS or a related disease. In other embodiments, the effective amount is the amount administered to a subject sufficient to reverse or slow the progression of signs or symptoms of ALS or a related disease. Assays that can be used to measure aggregation and the related activity of SOD dimer dissociation are described herein.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Superoxide dismutase” or “SOD”: The “superoxide dismuates” or “SOD,” as used herein, refers to any member of a family of different enzymes found in most living organisms. The term may refer to a superoxide dismutase protein or a polynucleotide encoding a superoxide dismutase protein. Variants, mutants, polymorphs, isotypes, and alleles of superoxide dismutase, which are at least 80%, at least 90%, at least 95%, at least 98%, or at least 99% homologous to wild type superoxide dismutase are also encompassed by this term. One function of superoxide dismutase protein is to destroy superoxide radicals (O₂⁻) Superoxide is a material naturally produced during phagocytosis and aerobic metabolism. The superoxide dismutases are characterized in families based on the metal ion associated with the enzyme, where the ions can be iron, manganese, copper, and copper and zinc. In certain embodiments, the superoxide dismutase is a human superoxide dismutase. In certain embodiments, the superoxide dismutase is SOD-1. The human Cu—Zn superoxide dismutase (SOD-1) is a dimeric protein composed of apparently identical noncovalently linked subunits, each with a molecular weight of 16,000-19,000 (U.S. Pat. No. 5,714,362; U.S. Pat. No. 5,629,189; Hartz, et al., J. Biol. Chem. 247:7043-7050 (1972); , Lieman-Hurwitz, et al., Biochem. Int. 3:107-115 (1981); Jabusch et al., Biochemistry 19:2310-2316; Barra et al., FEBS Letters 120:53-55 (1980); Lieman-Hurwitz et al., Proc. Natl. Acad. Sci. USA 79:2808-2811 (1982); each of which is incorporated herein by reference). The locus for human cytoplasmic superoxide dismutase (SOD-1) was assigned to chromosome 21 (Tan, et al., J. Exp. Med. 137:317-330 (1973); incorporated herein by reference).

### DETAILED DESCRIPTION OF THE INVENTION

I. Inhibitory Compounds

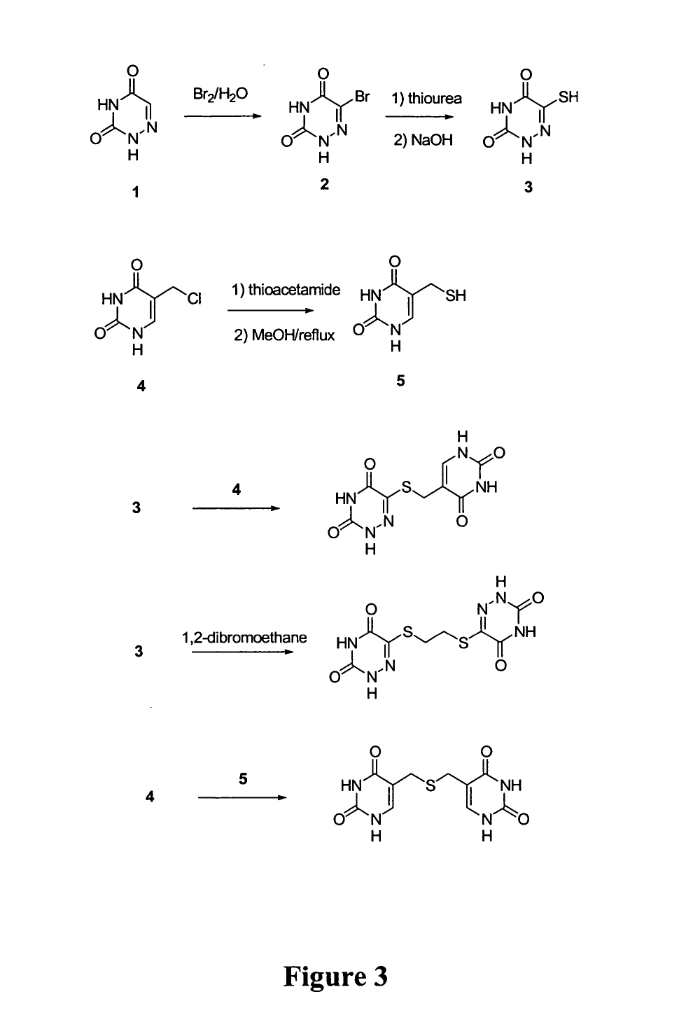

The ability of compounds to inhibit the aggregation of SOD can be determined using the procedures and assays described in the Examples section. Many of the individual compounds and classes of compounds described herein have been extensively studied and compounds can either be purchased commercially or synthesized using procedures well known in the art of organic chemistry. An exemplary procedures that can be used to prepare 1,2-di-[6-Mercapto-2H-[1,2,4]triazine-3,5-dione]ethane; and di-{5-[1H-pyrimi dine-2,4-dione]methyl}thioether are described in Example 3 and illustrated in FIG. 3.

Further, compounds of the invention may be designed in silico. The compounds are designed to bind the drug binding site at the interface of SOD dimers. The structure of SOD has been described in Deng et al. (Science 261(5124):1047-1051 (1993) which is incorporated herein by reference) and Hough et al. (Proc. Natl. Acad. Sci. USA 101(16):5976-81 (2004) which is incorporated herein by reference). The coordinates for the SOD structure have been deposited in a public database at www.rcsb.org/pdb and the accession number is 1spd.

The practitioner skilled in the art will appreciate that there are a number of ways to design compounds of the present invention. These same ways may be used to select a candidate compound for screening as an inhibitor of SOD aggregation. This design or selection may begin with selection of the various moieties which fill binding pockets.

There are a number of ways to select moieties to fill individual binding pockets. These include visual inspection of a physical model or computer model of the active site and manual docking of models of selected moieties into various binding pockets. Modeling software that is well known and available in the art may he used. These include QUANTA (Molecular Simulations, Inc., Burlington, Mass., 1992), and SYBYL (Molecular Modeling Software, Tripos Associates, Inc., St. Louis, Mo., 1992). This modeling step may be followed by energy minimization with standard molecular mechanics forcefields such as CHARMM and AMBER (AMBER: Weiner, et al., J. Am. Chem. Soc. 106:765 (1984); CHARMM: Brooks, et al., Comp. Chem. 4:187 (1983)).

In addition, there are a number of more specialized computer programs to assist in the process of optimally placing either complete molecules or molecular fragments into the binding site. These include: GRID (Goodford, J. Med. Chem. 28:849-857 (1985), available from Oxford University, Oxford, UK); MCSS (Miranker, et al., Proteins: Structure, Function and Genetics 11, 29-34 (1991), available from Molecular Simulations, Burlington, Mass.); DOCK (Kuntz, et al., J. Mol. Biol. 161:269-288 (1982), DOCK is available from the University of California, San Francisco, Calif.)

Once suitable binding orientations have been selected, complete molecules can be chosen for biological evaluation. In the case of molecular fragments, they can be assembled into a single inhibitor. This assembly may be accomplished by connecting the various moieties to a central scaffold. The assembly process may, for example, he done by visual inspection followed by manual model building, again using software such as Quanta or Syby1. A number of other programs may also be used to help select ways to connect the various fragments. These include: CAVEAT (Bartlett, et al, “CAVEAT: A Program to Facilitate the Structure-Derived Design of Biologically Active Molecules” In Molecular Recognition in Chemical and Biological Problems,” Special Pub., Royal Chem. Soc. 78: 182-196 (1989), CAVEAT is available from the University of California, Berkeley, Calif.), 3D Database systems such as MACCS-3D (MDL Information Systems, San Leandro, Calif., this area has been recently reviewed by Martin (J. Med. Chem. 35: 2145 (1992)) and HOOK (available from Molecular Simulations, Burlington, Mass.)

In addition to the above computer-assisted modeling of compounds, the compounds of this invention may be constructed “de novo” using either an empty active site or optionally including some portions of a known inhibitor. Such methods are well known in the art. They include, for example: LUDI (Bohm, J. Comp. Aid. Molec. Design. 6:61-78 (1992), LUDI is available from Biosym Technologies, San Diego, Calif.), LEGEND (Nishibata, Tetrahedron 47:8985 (1991), LEGEND is available from Molecular Simultations, Burlington, Mass.) and LeapFrog (available from Tripos Associates, St. Louis, Mo.)

A number of techniques commonly used for modeling drugs may he employed (For a review, see: Cohen, et al., J. Med. Chem. 33:883 (1990)). There are likewise a number of examples in the chemical literature of techniques that can be applied to specific drug design projects. For a review, see: Navia, et al., Current Opinions in Structural Biology 2:202 (1992)). Some examples of these specific applications include: Baldwin, et al., (J. Med. Chem. 32:2510 (1989); Appelt, et al., J. Med. Chem. 34:1925 (1991); and Ealick, et al., Proc. Nat. Acad. Sci. USA 88, 11540 (1991)).

Using the novel combination of steps of the present invention, the skilled artisan can advantageously reduce time consuming and expensive experimentation to determine dimer stabilization activity of particular compounds. The method also is useful to facilitate the rational design of compounds that prevent the aggregation of SOD and therapeutic or prophylactic treatments against neurological diseases such as ALS and related diseases. Accordingly, the present invention relates to such compounds.

A variety of conventional techniques may be used to carry out each of the above evaluations as well as the evaluations necessary in screening a candidate compound for activity in preventing SOD aggregation. Generally, these techniques involve determining the location and binding proximity of a given moiety, the occupied space of a bound compound, the amount of complementary contact surface between the compound and protein, the deformation energy of binding of a given compound and some estimate of hydrogen bonding strength and/or electrostatic interaction energies. Examples of conventional techniques useful in the above evaluations include: quantum mechanics, molecular mechanics, molecular dynamics, Monte Carlo sampling, systematic searches and distance geometry methods (Marshall, Ann. Rev: Pharmacol. Toxicol. 27:193 (1987)). Specific computer software has been developed for use in carrying out these methods. Examples of programs designed for such uses include: Gaussian 92, revision E.2 [M. J. Frisch, Gaussian, Inc., Pittsburgh, Pa., (1993)); AMBER, version 4.0 (Kollman, University of California at San Francisco, (1993)); QUANTA/CHARMM (Molecular Simulations, Inc., Burlington, Mass. (1992)); and Insight II/Discover (Biosysm Technologies Inc., San Diego, Calif. (1992)). These programs may be implemented, for instance, using a Silicon Graphics Indigo2 workstation or personal computer network. Other hardware systems and software packages will be known and of evident applicability to those skilled in the art.

Different classes of compounds, according to this invention, may interact in similar ways with the various binding regions of the SOD binding site. The spatial arrangement of these important groups is often referred to as a pharmacophore. The concept of the pharmacophore has been well described in the literature (Mayer, et al, J. Comp. Aided Molec. Design 1:3 (1987); Hopfinger, et al., in Concepts and Applications of Molecular Similarity: M. A. Johnson and G. M. Maggiora, Ed., Wiley (1990)).

Different classes of compounds of this invention may also use different scaffolds or core structures, but all of these cores will allow the necessary moieties to be placed in the active site such that the specific interactions necessary for binding may be obtained. These compounds are best defined in terms of their ability to match the pharmacophore, i.e., their structural identity relative to the shape and properties of the binding site of superoxide dismutase

Distances to or from any given group are calculated from the center of mass of that group. The term “center of mass” refers to a point in three-dimensional space which represents a weighted average position of the masses that make up an object. Distances between groups may be readily determined using any pharmacophore modeling software and other suitable chemical software. Examples of pharacophore modeling software that are commercially available include: DISCO (Martin et al. J. Comput. Aided Mol. Design 7:83 (1993), DISCO is available from Tripos Associates, St. Louis, Mo.); CHEM-X (Chemical Design Ltd., Oxon, UK and Mahwah, N.J.); APEX-3D (part of the Insight molecular modeling program, distributed by Molecular Simulations, Inc., San Diego, Calif.) CATALYST (Sprague, Perspectives in Drug Discovery and Design 3:1 (1995), Muller, Ed. ESCOM, Leiden, CATALYST is distributed by Molecular Simulations, Inc. San Diego, Calif.).

The binding pocket at the dimer interface includes residues Val7, Gly 147, and Val 148 from each subunit. The binding pocket also includes residues Gly56, ThrA54, AsnA53, LysA9, CysA146, ValA148, ValA7, GlyB51, Thr116, and Gly 147.

Compounds of the invention may be viewed as constituting four groups Group 1, 2, 3, and 4 in which Group 1 is a hydrophilic group; Group 2 is a hydrophilic group; Group 3 is a hydrophobic aromatic group; and Group 4 is a hydrophobic aromatic group. Group 1 is within about 5-6 Å from the center of the groups, preferably, about 5.25 Å; Group 2 is 4-5 Å from the center of the groups, preferably, about 4.75 Å; Group 3 is approximately 5 Å from the center of the groups; and Group 4 is about 3.5-4.5 Å, preferably approximately 4 Å, from the center of the groups. A given compounds may have two, three, or four of these groups. It will be appreciated that Group 1, Group 2, Group 3, and Group 4 or a subset thereof may be connected in various ways while satisfying the requisite distances described above.

In general, the binding regions for Groups 1 and 2 are hydrophilic. In certain embodiments, Group 1 and Group 2 include heteroatoms. Exemplary groups include hydroxy, amino, alkylamino, dialkylamino, thioxy, alkyoxy, alkylthioxy, carbonyls, esters, amides, carbonates, ureas, carbamates, aldehydes, ether, thioethers, nitroso, halogen, hydrazine, hydrazide, phosphate, carboxylic acid, acyl, heteroaryl, heteroaliphatic, cyano, and isocyano. Amino acids Gly56, ThrA54, AsnA53, LysA9, CysA146, ValA148, ValA7, GlyB51, Thr116, and Gly147 form the binding pocket for Group 1, and the two-fold symmetry related amino acids form the binding pocket for Group 2.

In general, the binding regions for Groups 3 and 4 are hydrophobic. In certain embodiments, Group 3 and/or Group 4 is a substituted or unsubstituted aryl group. In certain embodiments, Group 3 and/or Group 4 is a substituted or unsubstituted heteroaryl group. Exemplary groups that may be introduced as Group 3 and Group 4 include phenyl, naphthalene, anthracene, phenanthrene, toluene, mesitylene, triphenylmethane, benzaldehyde, O-benzyl, benzoic acid, phenyl methyl ether, nitrophenyl, pyridyl, pyrimidinyl, pyrrolindinyl, tetrahydrothiophene, tetrahydrofuran, piperidinyl, pyranyl, dioxanyl, morpholinyl, pyrrolyl, thiophene, furanyl, pyrazinyl, triazinyl, imidazolyl, thiazolyl, oxazolyl, indolyl, purine, pyrone, pyridone, quinoline, and isoquinoline. In certain embodiments, Group 3 and/or Group 4 is a monocyclic ring system. In certain embodiments, Group 3 and/or Group 4 is an aromatic five- or six-membered ring optionally substituted. In certain particular embodiments, Group 3 and/or Group 4 is a phenyl ring optionally substituted. In certain embodiments, Group 3 and/or Group 4 is a substituted phenyl moiety. Amino acids Val5, Cys6, Val7, Leu8, Lys9, Gln15, Gly 16, and Ile17 form the binding pocket for Group 3, and the same amino acids on the other subunit form the binding pocket for Group 4.

Linker moieties useful in covalently attaching the various group together may include substituted or unsubstituted, cyclic or acyclic, branched or unbranched aliphatic or heteroaliphatic groups. In certain embodiments, the linker is rigidified for better binding of the groups in the binding site. For example, the linker may include cyclic structures, the linker may include substitutions such as methyl group, the linker may include various degrees of unsaturation, etc. In other embodiments, the linker is flexible. Exemplary linker groups include:

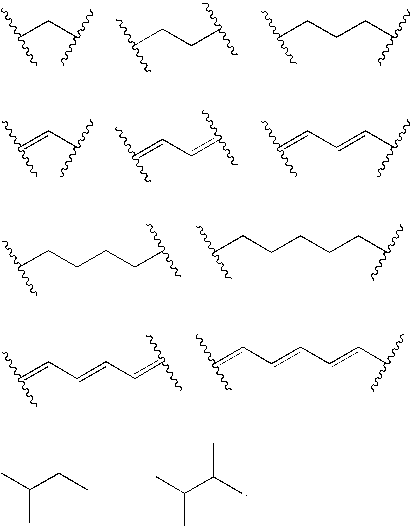

II. Drug Formulations

The present invention is compatible with the delivery of compounds by any means known in the art, including peroral, internal, rectal, nasal, lingual, transdermal, intravenous, intraarterial, intramuscular, intraperitoneal, intracutaneous and subcutaneous routes. The most preferred route is oral (especially using dosage forms such as tablets, capsules or solutions). It may also be desirable in some instances to administer compounds directly into the cerebrospinal fluid of patients.

Guidance in preparing pharmaceutical formulations for a compound may be obtained from compositions used for similar compounds that are commercially available and from descriptions in the art. It will also be appreciated that compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference.

The term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

The pharmaceutical compositions of the present invention may additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/druglistframe).

In certain embodiments, the pharmaceutical compositions of the present invention further comprise one or more additional therapeutically active ingredients (e.g., chemotherapeutic and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

III. Treatment Methods

Test animals and subjects (e.g., human subjects) diagnosed as having ALS or a related disease may be treated by administering one or more of the compounds described above. The exact dosage will depend upon the particular compound being administered and will be determined using procedures well known in the art, balancing toxicity and therapeutic efficacy. In the case of patients, dosages will typically be adjusted by the attending physician based upon clinical conditions with the therapeutic objective of slowing the decline in nerve and muscle function. When compounds are given to test animals to study the effect of inhibiting SOD aggregation, dosage can be titrated over a wide range and is limited only by toxicity.

Although the main contemplated use of the compounds is in the study and treatment of ALS, it should be recognized that any other disease or condition that is associated with SOD aggregation may also be treated. In particular, the compounds may be used to treat other neurological disorders, e.g., Alzheimer's disease, and cancers. Several of the compounds found to be active in inhibiting SOD aggregation may be administered together or they may be given as the sole active agent. Compounds may also be combined with other treatment methods to improve overall effectiveness. Once initiated, treatment should typically continue throughout the life of a patient.

IV. Fluorescent Assay for Measuring SOD Aggregation

A. Excimer Assay

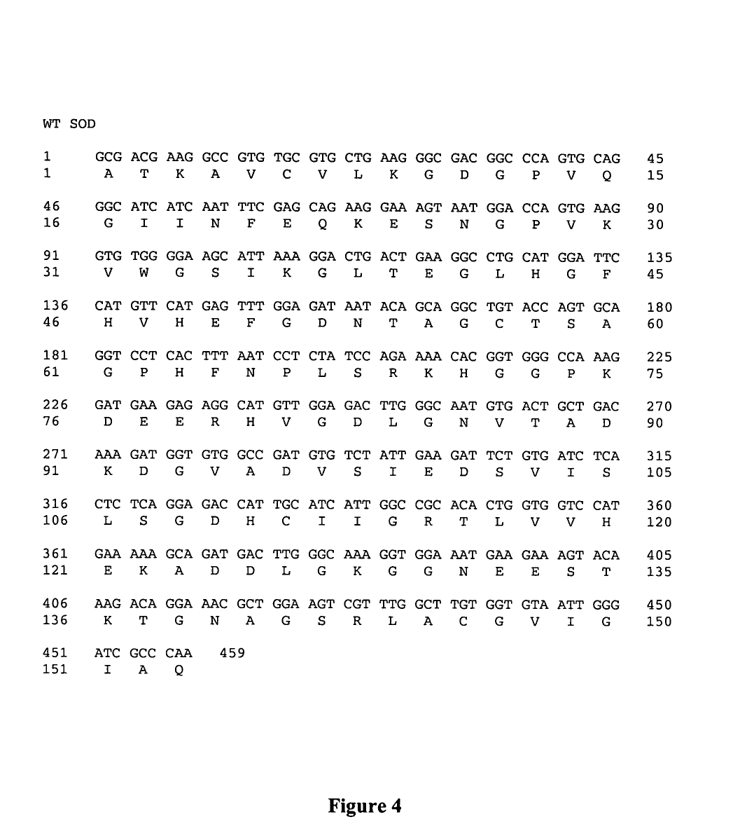

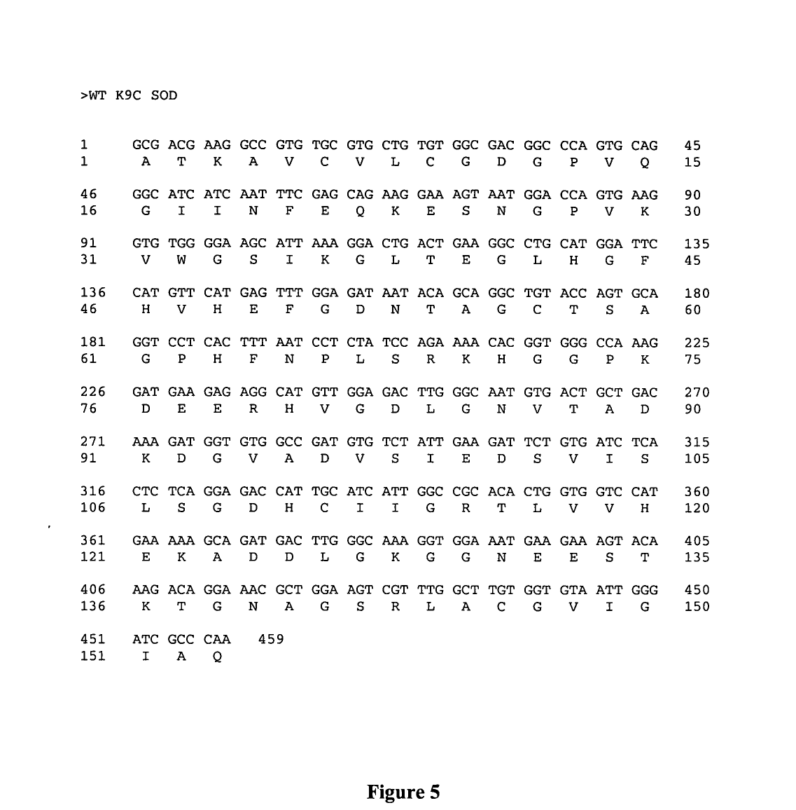

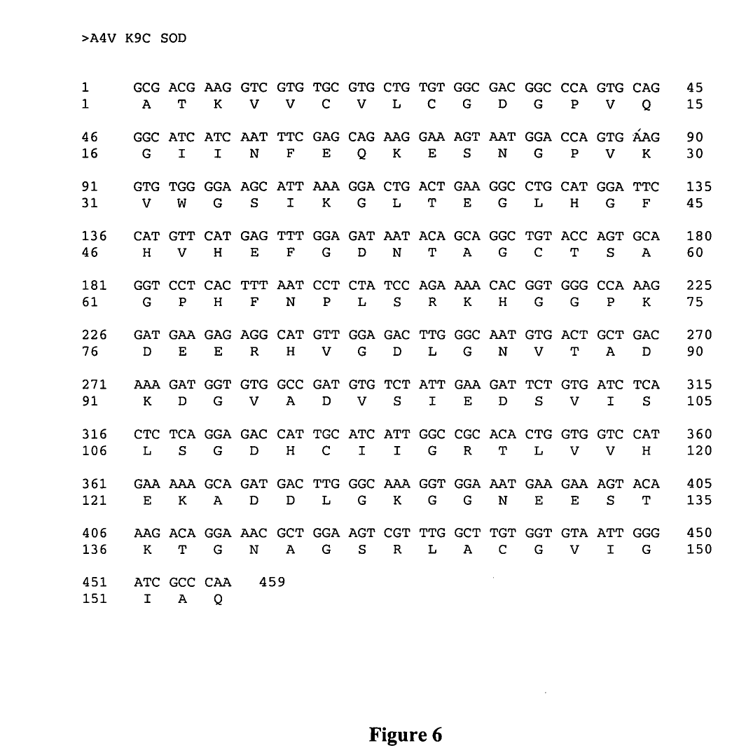

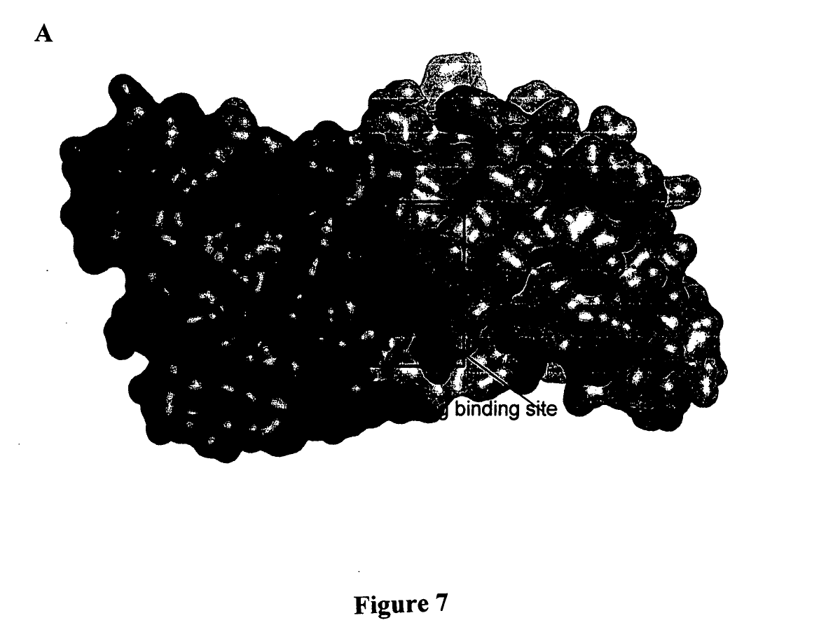

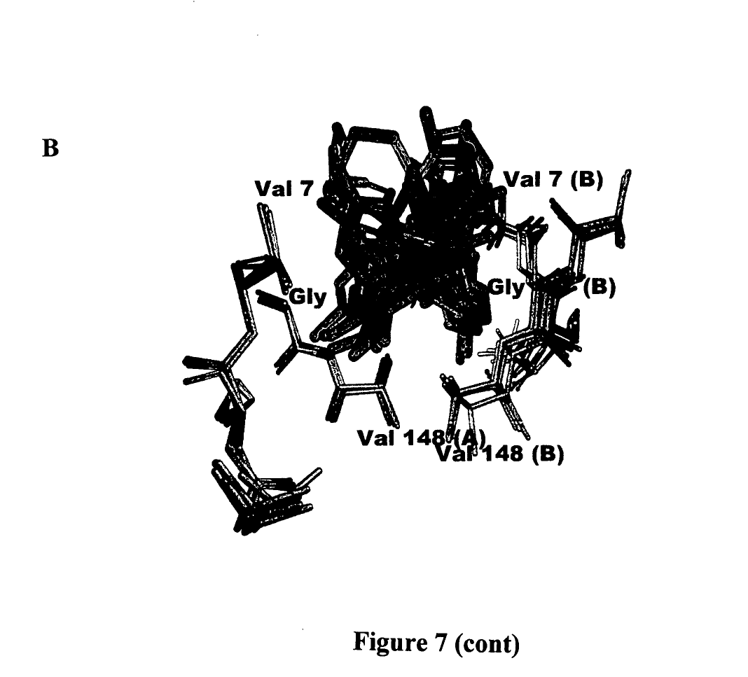

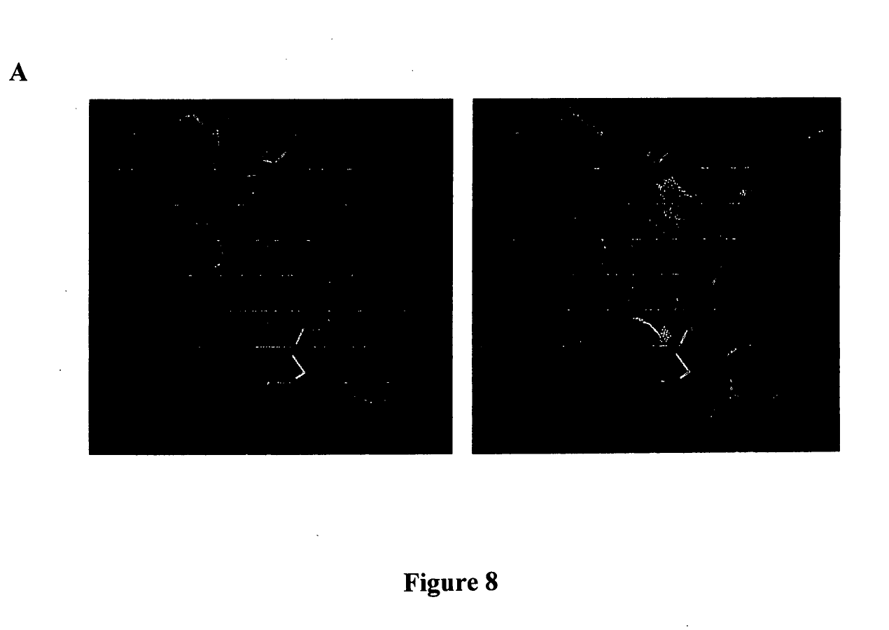

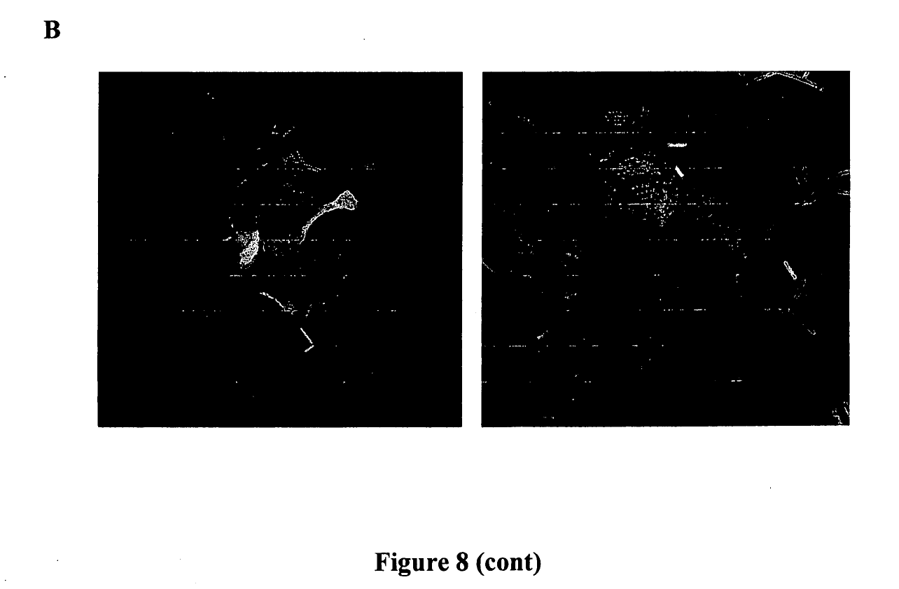

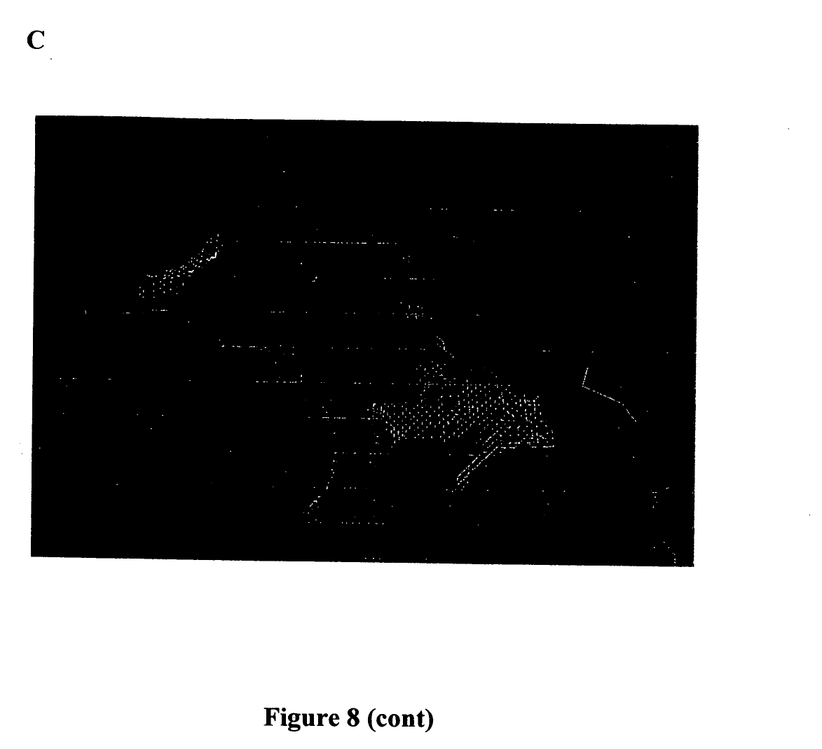

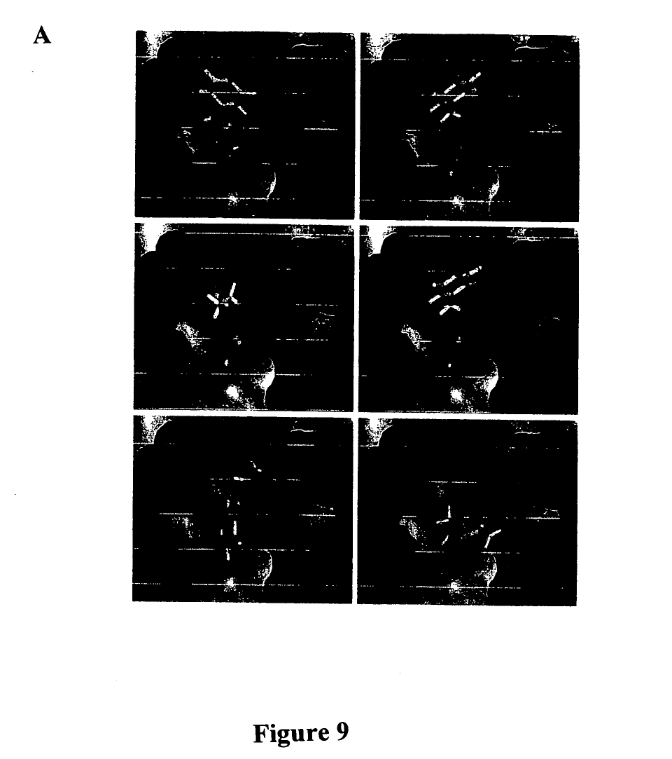

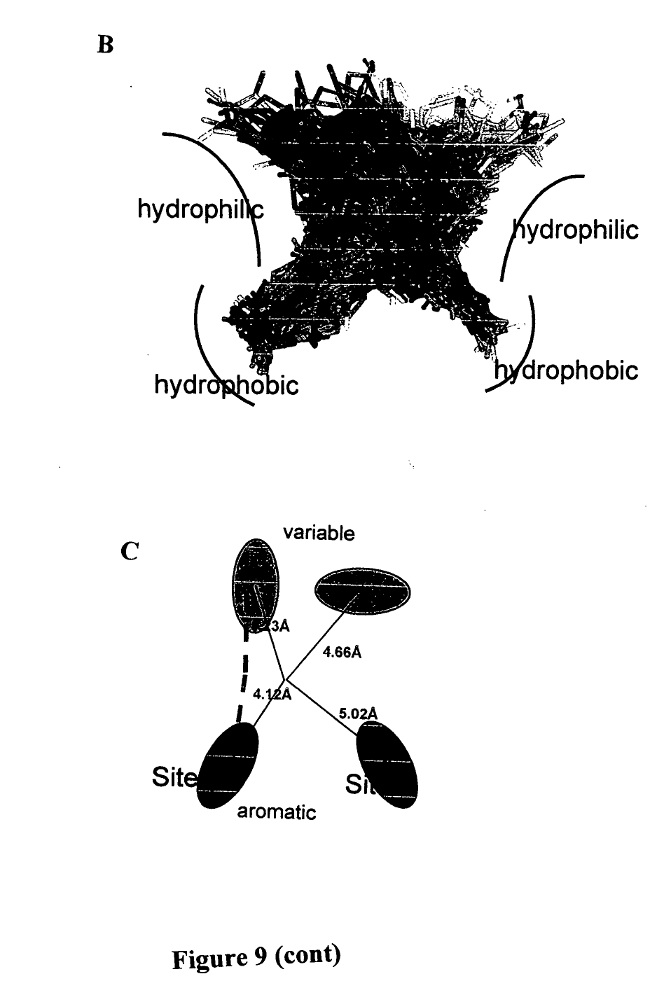

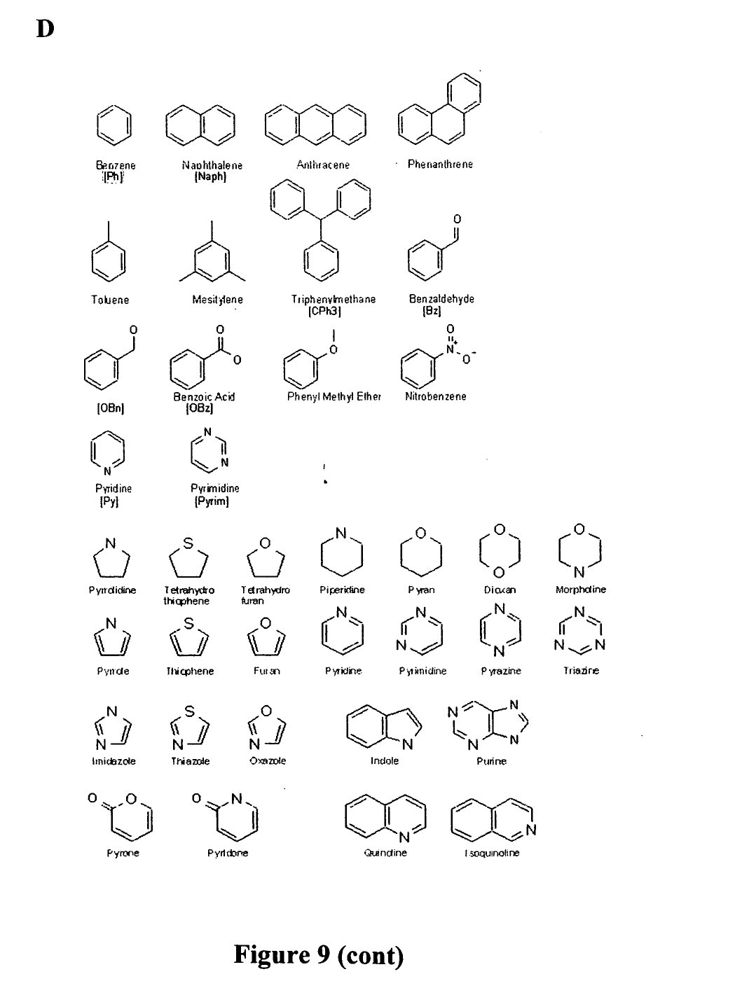

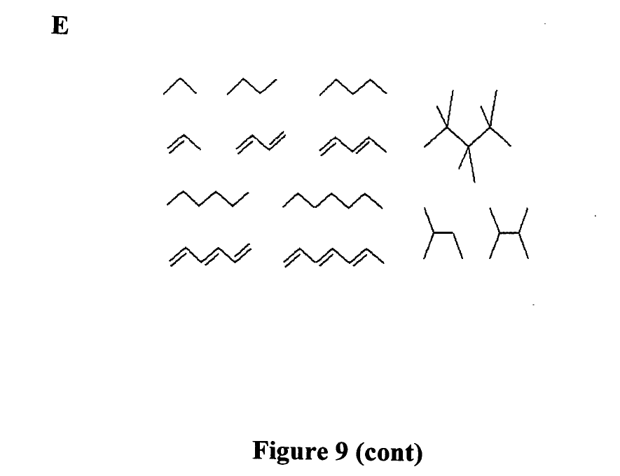

The invention also includes fluorescence-based excimer assays that measure SOD1 dimer stability. These assays involve fluorescently labeling an amino acid in SOD that is found at the interface of dimers. Thus, upon dimer formation fluorescent labels are brought into close proximity to one another and form an excimer. Using the SOD sequence shown in FIG. 4 (SEQ ID NO:1) as a reference, suitable amino acids appear to be Gly51; Asp52; Thr54; Ala55; Ser59; Ala1; Thr2; Ala4; Val5; Val7; Lys9; Gly10; Asp11; Gly12; Gln15; Ser107; Gly108; Asp109; Cys111; Ile113; Gly114; Arg115; Thr116; and Leu117. Many procedures are known in the art that can be used for labeling and, depending upon the strategy adopted and the fluorophore chosen, amino acids at these sites may be altered. Examples of fluorescent labels that can be used include fluorescein, rhodamine and pyrene. However, other fluorophores known in the art can also be used.

In a preferred embodiment Lys9 is mutated to Cys and modified with pyrene. Dimer formation allows the formation of the pyrene excimer which is observable based on its long wavelength absorption (ca. 490 nm). Thus, by labeling SOD, isolating dimers and incubating them in the presence of a large excess of unlabeled SOD, the rate of dimer dissociation can be determined by measuring time-dependent formation of heterodimer, in which only one subunit is labeled. By way of example, A4V/K9C dimers produce an excimer band at 490 nm. On dilution with excess A4V, the intensity of the excimer absorption decreases, as the absorption due to the monomeric pyrene (390 nm) increases.

B. FRET Assay

Fluorescence resonance energy transfer (FRET) assays may also be used to identify compounds stabilizing SOD dimers. FRET is a phenomenon by which two fluorophores (one an energy acceptor and one an energy donor) that are located within ca. 100 Å of one another, can exchange energy. If the emission wavelength of one fluorophore matches the excitation wavelength of the other (and they are close in space), FRET is observed. This is a convenient and widely used method to assay protein dimerization. Examples of fluorophore donor/acceptor pairs are: pyrene/perylene and fluorescein/rhodamine.

The FRET method may be used to observe the kinetics of dimer dissociation. Compounds that slow the rate of dissociation should also shift the monomer-dimer equilibrium towards the dimer. The FRET-optimized fluorophores (A488 LexA and A594 Rhodamine) may be introduced by chemical modification of A4V/K9C using standard methods to form enzyme with an attached donor fluorophore group, SODD, and enzyme with an attached acceptor fluorophore group (SODA). The A488 dimer produces only the predicted emission at 550 nm. However, when mixed with an equal amount of the A594 dimer, that emission is greatly reduced and the FRET emission at 630 nm appears, indicating the formation of heterodimers.

To screen compounds for those that stabilize dimers, each modified SOD1 is purified in homodimeric form. After mixing (SODD)₂ and (SODA)₂ in a 1:1 ratio and allowing for equilibration/subunit exchange, a FRET signal should be observed due to the (SODA)(SODD) heterodimer (the two homodimers, (SODD)₂ and (SODA)₂, will not produce a FRET signal and will be ignored). With the heterodimer FRET signal as baseline, a large molar excess of unlabelled SOD1 is added and the rate of disappearance of the FRET signal is measured. In general, conditions should be optimized so that a complete loss of signal occurs in about 12 hours. For high throughput screening, compound is added with unlabelled SOD1 and the rate of signal loss in the presence of the compound is compared with that in its absence. Compounds that stabilize dimers will be those that slow the loss of signal. This assay has the advantage that only hits will produce a signal. In addition, it is not necessary to apply an “unnatural” demetallating reagent to observe dissociation.

### EXAMPLES

### Example 1

### Assays and Initial Screening of Compounds

The present example is concerned with a strategy for inhibiting SOD-1 aggregation based upon stabilization of the SOD-1 native dimer with small, drug-like molecules (15). This strategy is based upon the concept that SOD-1 monomerization is required for aggregation, which is supported by the observation that insertion of an engineered intersubunit disulfide bond into the FALS SOD-1 mutant A4V prevents its aggregation (16). The proposal that monomerization of the protein is required for in vivo aggregation is also supported a detailed analysis of the aggregation of SOD-1 (10).

Precedent for the discovery and use of small-molecule stabilizers of a native protein oligomer may be found in connection with a protein aggregation disease that is analogous to FALS: familial amyloid polyneuropathy (FAP). FAP is caused by mutations in the gene encoding transthyretin (TTR) (17, 18). Many FAP mutations destabilize the native TTR tetramer, facilitating its dissociation, partial unfolding, and aggregation (17, 19). The natural ligand of TTR, thyroxine, stabilizes the tetramer and prevents its aggregation in vitro. Drug-like molecules that are thyroxine analogs also bind and stabilize the native TTR tetramer, preventing its aggregation in vitro (20-24). These compounds could potentially be used for the treatment of FAP (25).

Unlike the example of TTR, we know of no natural ligands of SOD-1 to serve as a molecular scaffold for the design of small-molecule stabilizers. Therefore, we decided to take an in silico screening approach (docking), using a library of approximately 1.5 million drug-like molecules, to select for compounds that could potentially bind at the dimer interface. In the present example, fifteen compounds are identified by this method that have the ability to significantly stabilize A4V (and other FALS variants) and prevent aggregation.

A. Materials and Methods

Cloning and Purification

Cloning, expression and purification of human SOD-1, WT and the various FALS and other mutants described in the investigation is carried out as described previously (16).

Database Preparation and Docking

All computations were carried out on an 18 node Beowulf Linux Cluster (each node=2.0 GHz Pentium processor). Raw structure data files obtained from vendors were filtered to remove wrong structures. Database preparation and docking were carried out using a trial version of Schrödinger's First Discovery Suite, which included GLIDE v2.5 being the primary tool for docking (26).

Purification of Recombinant SOD-1 Dimer and Metal Analysis

SOD-1 dimer was purified on a Superdex 75™ (16/60) gel filtration column (Pharmacia) to produce starting material for each aggregation experiment. Metal analyses were carried by inductive coupled plasma mass-spectrometry (ICP-MS). WT and G93A were nearly fully metallated, and G85R and A4V were deficient in zinc and copper, respectively.

Preparation of Apo-SOD-1 and Variants

The procedure of Fridovich and coworkers (27) was followed, with minor modifications. The loss of Cu and Zn were confirmed using ICP-MS analysis; all variants prepared in this way contained less than 0.2% of Cu or Zn.

Aggregation of SOD-1 and Mutants

Aggregation assays for screening were prepared by adding a stock solution of compound to a protein solution (final concentrations: 100 μM compound, 50 μM protein). Following a 15 minute preincubation period at 37° C., 5 mM EDTA was added to initiate aggregation. Aliquots were periodically removed and analyzed for the amount of SOD-1 dimer present. This value correlated in all cases with the appearance of oligomers by gel filtration on a Superdex 200™ (3.2/30) gel filtration column (Pharmacia). All chromatography was performed in TBS, pH 7.4 (20 mM Tris, 150 mM NaCl) on a Waters 2690 Alliance HPLC and monitored at 220 and 276 nm. The assays were repeated in triplicate and showed less than 5% variation between individual experiments. Assays in the absence of EDTA were carried as described previously (16). For Apo-A4V experiments, buffers were treated with CHELEX (except those containing EDTA) and experiments were performed in plastic tubes to avoid introduction of contaminating zinc into the Apo-protein.

Guanidinium chloride Unfolding

Equilibrium unfolding transition, as a function of GdnCl concentration, was monitored by fluorescence spectroscopy. The fluorescence measurements were done on a Hitachi f-4500 spectrofluorometer in a 1-cm cell connected to a circulation water bath. The excitation and emission wavelengths were fixed at 278 and 348 nm, respectively, after making appropriate corrections for buffer and GdnCl. The slit width was 5 nm for both monochromators. Each measurement was an average of five readings. Protein concentration used for fluorescence experiments was 5 μM. The data were analyzed directly for a two-state (N→U) transition as follows: the raw data for the GdnCl-induced denaturation studies were converted to fractions of the protein in the unfolded state (fu) as a function of GdnCl concentration using the equation:
fu=Y0−(Yf+mf[GdnCl.])/(Yu+mu[GdnCl.])−(Yf+mf[GdnCl.])
where Y0 is the observed spectroscopic property, Yf and mf are the slope and intercept of the folded state baseline and Yu and mu represent the respective values of the unfolded baseline. The folded fraction was calculated as (fn=1−fu) and the equilibrium constant was determined by Keq=fu/fn. The free energy of unfolding was determined using the equation ΔG=−RT ln(Keq) where T is the temperature in Kelvin and R is the universal gas constant (1.987 cal mol−1 K−1).

Aggregation of α-synuclein

Samples of α-synuclein were dissolved in PBS, pH 7.4, and filtered through a Millipore Microcon 100K MWCO filter. Samples were incubated at 37° C. without agitation. A 100 μM aqueous solution of Thioflavin T (Thio T; Sigma) was prepared and filtered through a 0.2 μm polyether sulfone filter. At various time points, aliquots of the α-synuclein incubations were diluted to 10 μM in water. Fluorescence measurements for the 300 μM αsynuclein incubations were performed in a 384-well microplate as described previously (28). Fluorescence at 490 nm was measured using the LJL Biosystems plate reader (excitation: 450 nm, bandwidth 30 nm; emission: 490 nm, bandwidth 10 nm).

B. Results

Filling a Hydrophobic Cavity at the A4V SOD-1 Dimer Interface Stabilizes it Against Unfolding and Aggregation

In order to look for suitable binding sites for small molecules at the SOD-1 dimer interface, we used the program VOIDOO (Uppsala software factory), which detects cavities in proteins (29). Five cavities were detected by the program, one of which was at the dimer interface of both WT and A4V. The cavity is centered with the Cβ carbon of residue 148 as the point of origin. The site is predominantly hydrophobic in nature with a few hydrogen bond donors and acceptors.

To investigate the effect of partially capping the cavity with hydrophobic moieties, residues V148 and V7, the sidechains of which protruded into the cavity, were mutated to phenylalanine. Molecular modeling suggested that the four Phe residues at the interface could be easily accommodated, with no steric clashes. Filling cavities in protein structures with hydrophobic side chains often stabilizes the protein structure (30, 31), lysozyme being a classic example (32).

Three variants of SOD-1, in which the V7F and V148F mutations were introduced into WT, A4V and G93A, were cloned, expressed in E. coli and purified as described previously (15). Each protein was subjected to guanidine chloride (GdnCl) unfolding and fluorescence intensity (348 nm) was monitored at 25° C. WT enzyme was completely unfolded at 3.5 M GdnCl (Cm=3.2 M, where Cm is the midpoint of transition), while A4V was completely unfolded at 1.9 M GdnCl (Cm=1.51 M). A4V/V7F/V148F was found to be more stable compared to A4V but less stable than WT (unfolded at 2.1 M, Cm=1.8 M). G93A/V7F/V148F was slightly more resistant to denaturation than G93A. No significant effect of the two V→F mutations on the denaturation of WT (i.e., WT vs. V7F/V148F) could be measured. The V→F mutations stabilized both A4V and G93A against EDTA induced aggregation. A4V/V7F/V148F aggregated more slowly than A4V but significantly faster than WT. Similarly, G93A/V7F/V148F aggregated slightly more slowly than G93A.

Preparation of a Compound Database and High-Throughput Docking of Compounds to the Cavity at the A4V Interface

An in silico screening approach was undertaken to identify compounds from commercially-available databases with a potential to bind at the SOD-1 dimer interface and stabilize the dimer. Pre-filters were used to select a subset of compounds that are more suited towards a particular target. Structure Data files (SD file) for 15 commercially available libraries were gathered.

Docking calculations were carried out using a trial version of Schrödinger (www.schrodinger.com) software, GLIDE v2.5 (26). The docking calculation has 2 distinct steps: (1) Docking of ligands; and (2) Scoring of hits. The protein structural data file for A4V (1UXM.pdb) was used for all calculations. A primary grid box of 7 Å and a secondary ligand containment box were generated around the Cβ carbon of residue 148 for the docking calculation of the protein after removal of water and addition of hydrogen atoms as the center of mass.

A detailed description of the GLIDE methodology has been published (26, 34). The molecules obtained after docking were analyzed and sorted by glidescores (26, 34). The top 100 binders were examined and it is noteworthy that approved drugs such as baclofen, dapsone and trimacicolone were among these. Superposition of x-ray structures of WT, apo-WT, S134N, H46R and A4V (pdb codes: 1spd, 1h14, 1ozu, 1oez and 1uxm) reveal very low r.m.s.d (<0.6 Å for Cα) between residues that make up the binding pocket in these variants as compared to A4V, suggesting that compounds are likely to bind to several mutants.

Fifteen of the Top One Hundred in Silico “Hits” Significantly Inhibited A4 V Aggregation

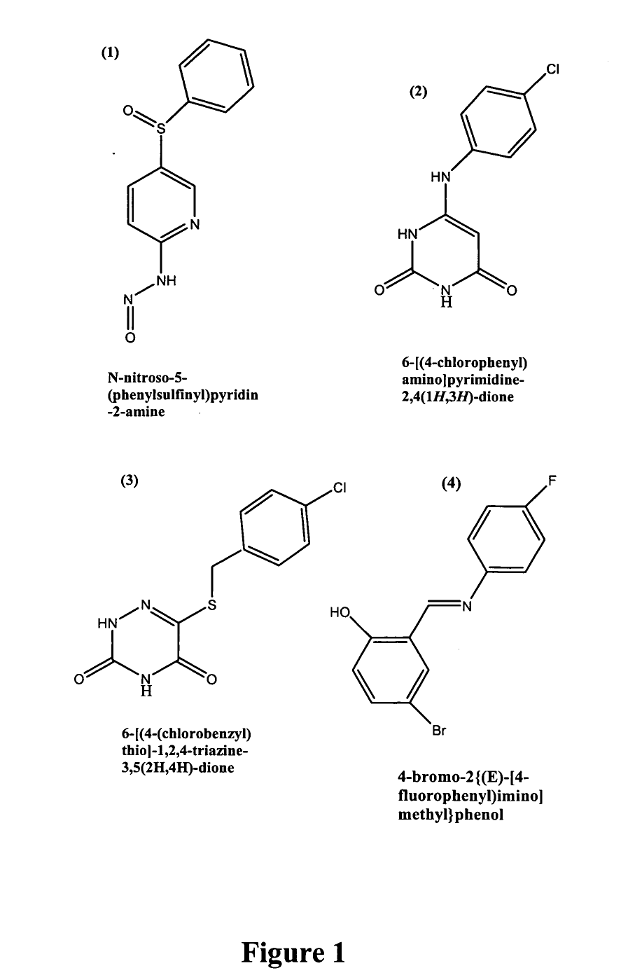

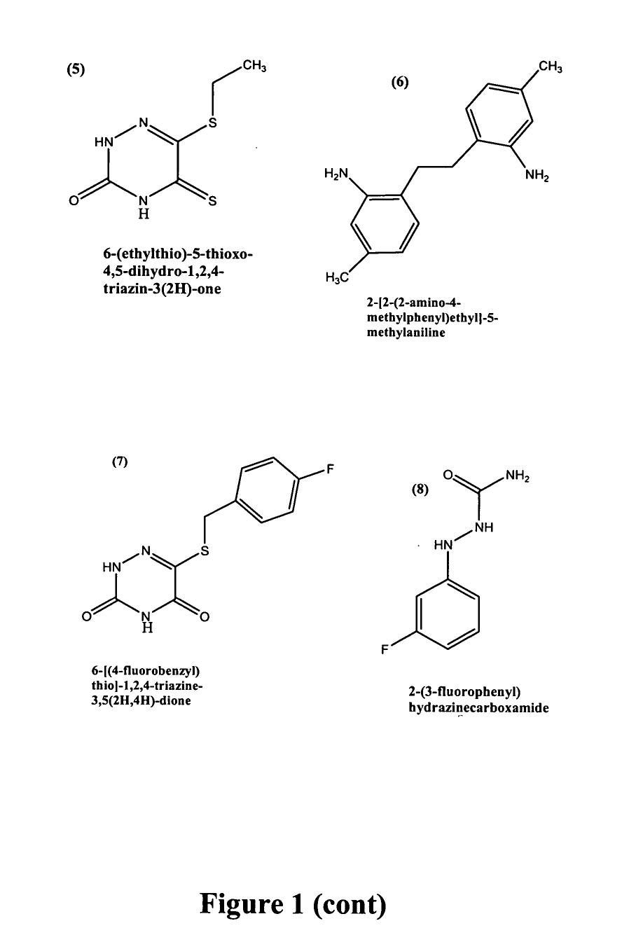

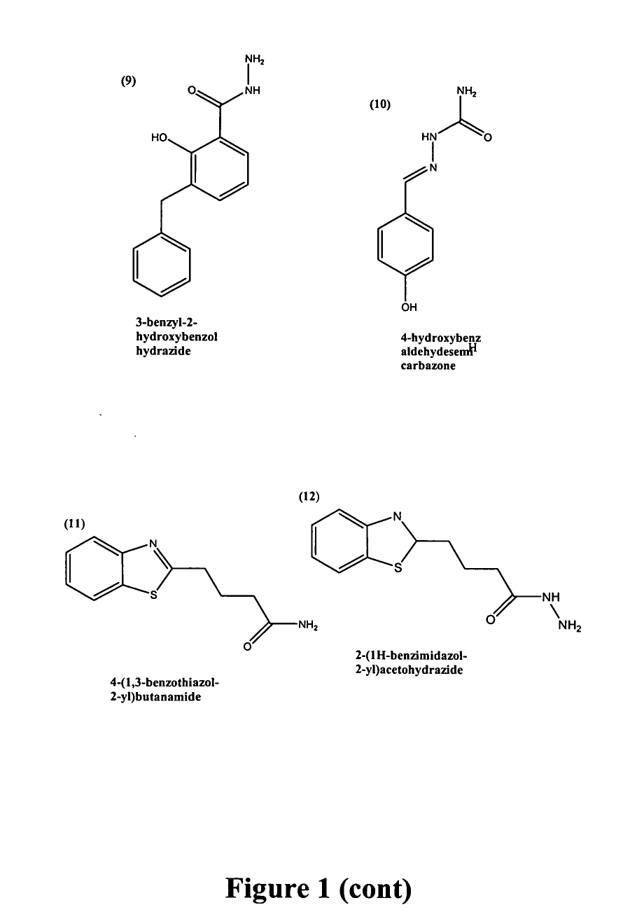

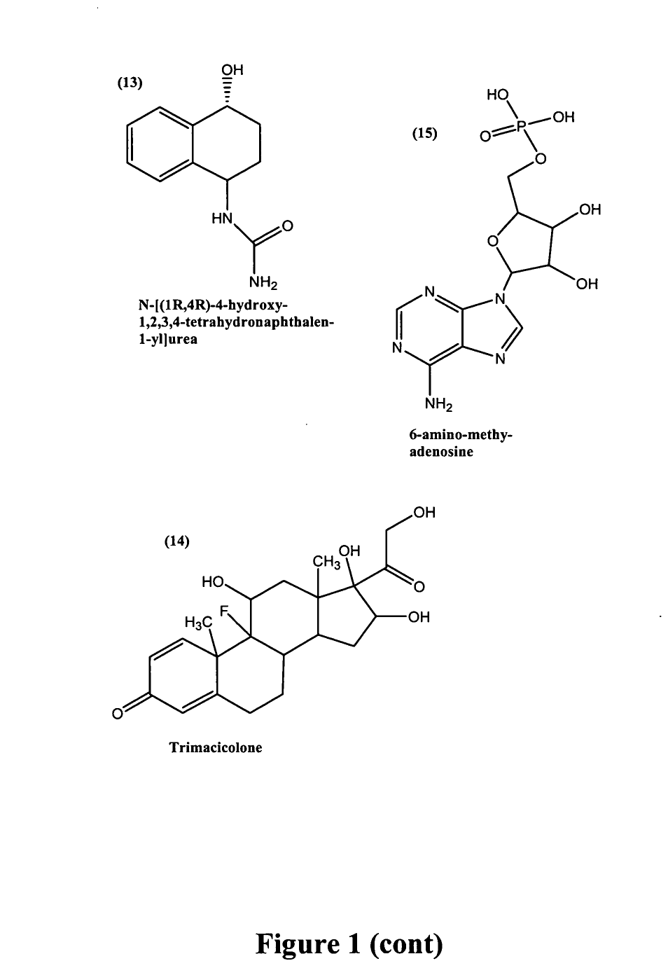

A4V aggregation assays (with EDTA, see Materials and Methods) were carried out in presence of the top 100 hits obtained as described above. The effect of each compound was compared to A4V and to WT in the absence of added compounds. Approximately 15 of the top 100 compounds significantly slowed A4V aggregation; that is, in the presence of these compounds, <25% of the dimer had disappeared after 12 h, whereas 50% was lost in their absence (these compounds, arbitrarily numbered 1-15, are shown in FIG. 1). In the presence of several of these compounds, A4V aggregation closely resembled WT (ca. 5% dimer loss after 12 h). The shape of the A4V aggregation curve may reflect the heterogeneity of the protein with respect to metallation: the initial rapid phase may represent the population lacking copper (apo-A4V does not show this “bi-phasic” behavior).

The Inhibitory Effect was Independent of Metal Binding Site Occupancy

Since the aggregation assay described above utilized EDTA to promote metal loss and accelerate aggregation (8), the observed inhibitory effect of a given compound could have been due to inhibition of demetallation, rather than inhibition of dimer dissociation. In order to rule out the former possibility, the effects of compounds 1-15 on the aggregation of A4V in the absence of EDTA were measured. It was found that all 15 compounds slowed aggregation of A4V under these conditions. All 15 compounds also inhibited the aggregation of the completely demetallated apo-A4 (also in the absence of EDTA). This effect suggests that these molecules can bind and stabilize the apo-A4V dimer (crystalline apo-WT and metallated WT are indistinguishable with respect to the cavity that is the focus of our screen).

The Inhibitory Effect of These Compounds is Likely to be Due to their Affinity for the Cavity at the A4 V Interface

To validate the rationale behind our in silico screening approach, we tested four of the most potent A4V aggregation inhibitors (2, 3, 4, and 7) to determine whether they were capable of inhibiting the aggregation of A4V/V7F/V148F, where the putative binding site had been disturbed. There was no appreciable change in the aggregation rate of A4V/V7F/V148F in presence of these compounds. Furthermore, a set of 20 arbitrarily chosen compounds from the initial database were found to have no effect on the aggregation of A4V. Finally, none of the top fifteen A4V inhibitors affected the aggregation of α-synuclein.

The A4V Aggregation Inhibitors also Inhibited Aggregation of other FALS Linked SOD-1 Mutants

Since the cavity at the A4V dimer interface is conserved in other FALS-linked SOD-1 mutants (see above), we expected that the A4V inhibitors may also inhibit the aggregation of these proteins. Aggregation (EDTA-induced) of both G93A and G85R were significantly inhibited by several of the A4V inhibitors. Interestingly, compounds 2, 3, 4 and 7 were among the best inhibitors in each case, as was the case with A4V. WT SOD-1 was also subjected to aggregation in presence of these compounds under the same conditions as described above. The aggregation of WT SOD-1 under these conditions was too slow to observe a significant inhibition.

All Fifteen A4V Aggregation Inhibitors also Stabilized A4V Against Denaturation

If the A4V aggregation inhibitors act by binding A4V dimer and inhibiting its dissociation, they should also stabilize the native dimer against chaotrope induced unfolding. Although the completely unfolded state is probably not relevant to the aggregation pathway (10), these experiments provide a convenient and well-precedented method to measure the relative stability of the native A4V dimer in the presence and absence of small molecules. All fifteen of the aggregation inhibitors significantly protected A4V from GdnCl induced unfolding. As controls, ten of the eighty-five compounds that did not show significant aggregation inhibition were tested and none of these had a significant effect on unfolding. The unfolding curves were analyzed directly, assuming a two-state (N→U) transition, and thermodynamic properties were measured by fitting the data to a linear extrapolation model (35). The stabilization of A4V in presence of the compounds was expressed as ΔG values. These values reflect the binding energy of the compounds, presumably to the cavity at the dimer interface. Four compounds (2, 3, 4 and 7) stabilized A4V nearly to WT levels. These four compounds were among the most potent aggregation inhibitors.

C. Discussion

The aggregation of mutant forms of SOD-1 may be pathogenic in FALS. This process is very complex, even under controlled in vitro conditions, since it may require loss of copper and zinc, reduction of an intrasubunit disulfide, monomerization, and partial unfolding (7,9-14). The work here was based on the premise that stabilization of the SOD-1 native dimer will inhibit its aggregation regardless of the exact pathway, since it will deplete the population of the aggregating species, which may be a partially unfolded apo-monomer. It is very important to note that one product of this simple screen (compound 2), even without optimization by medicinal chemistry, stabilizes the A4V dimer to an extent comparable to the difference in stability between the invariably lethal A4V and WT SOD-1.

The objective of the in silico screen, which was to select an easily screenable compound set that would have a high likelihood of binding to the A4V dimer, was met: 15 of our top 100 hits had significant activity in experimental assays for A4V aggregation and A4V unfolding. Several control experiments support the proposal that these compounds are binding to the cavity at the A4V interface, the intended mechanism of action. Of course, some of the compounds that were not in the top 100 may have activity (although 20 randomly-chosen compounds form the original library had no activity).

The group of drug-like compounds that are reported here are chemically and structurally similar (FIG. 1). Modeling of the interaction of these compounds with the A4V dimer interface show that the shared aromatic moiety may occupy the space between the two Val 148 residues of the SOD-1 subunits (introduction of an intersubunit disulfide at this position, by mutagenesis, was shown to stabilize the dimer of A4V against aggregation (16)).

### Example 2

### Testing of Structural Analogs

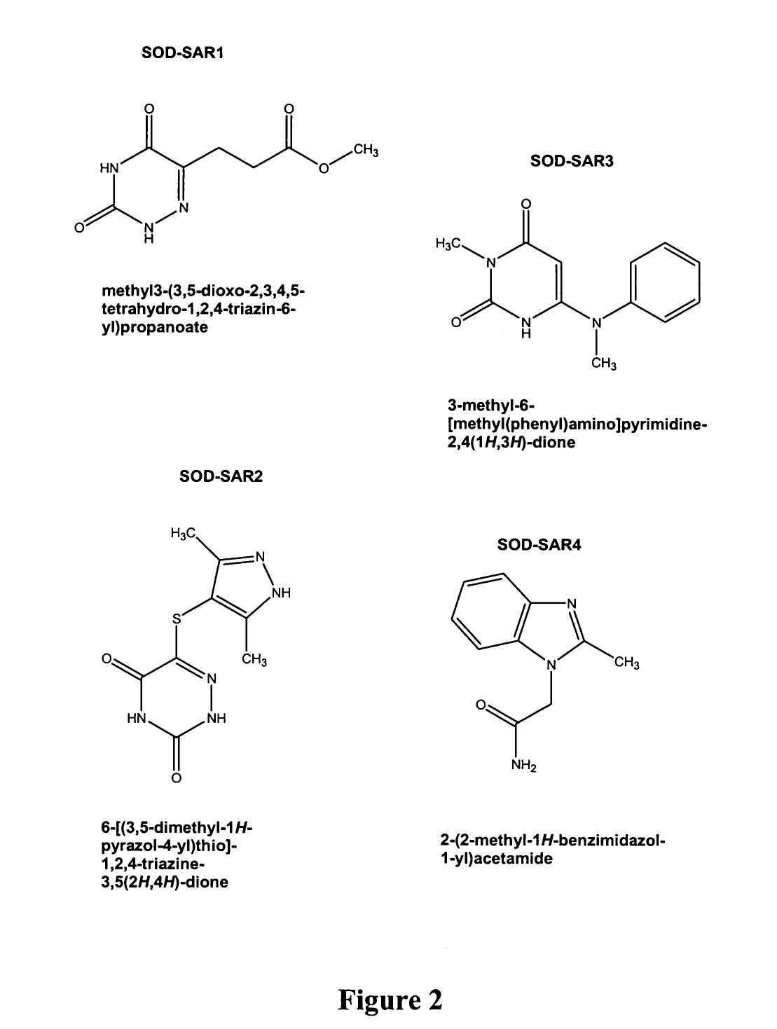

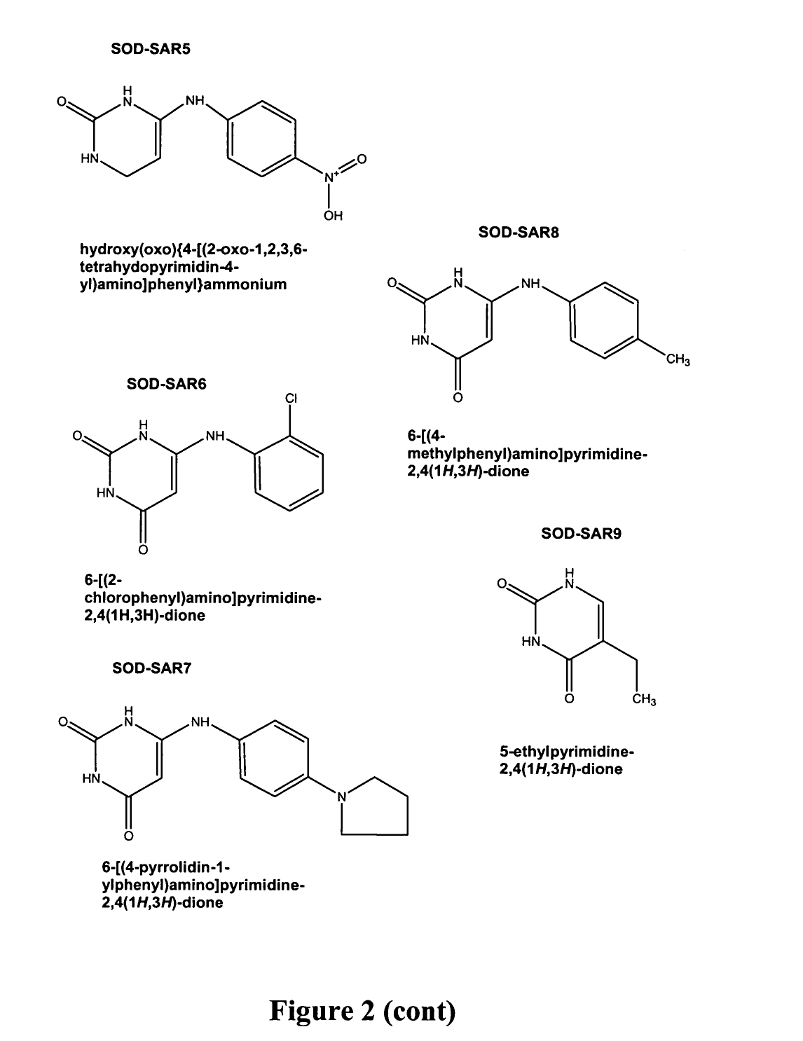

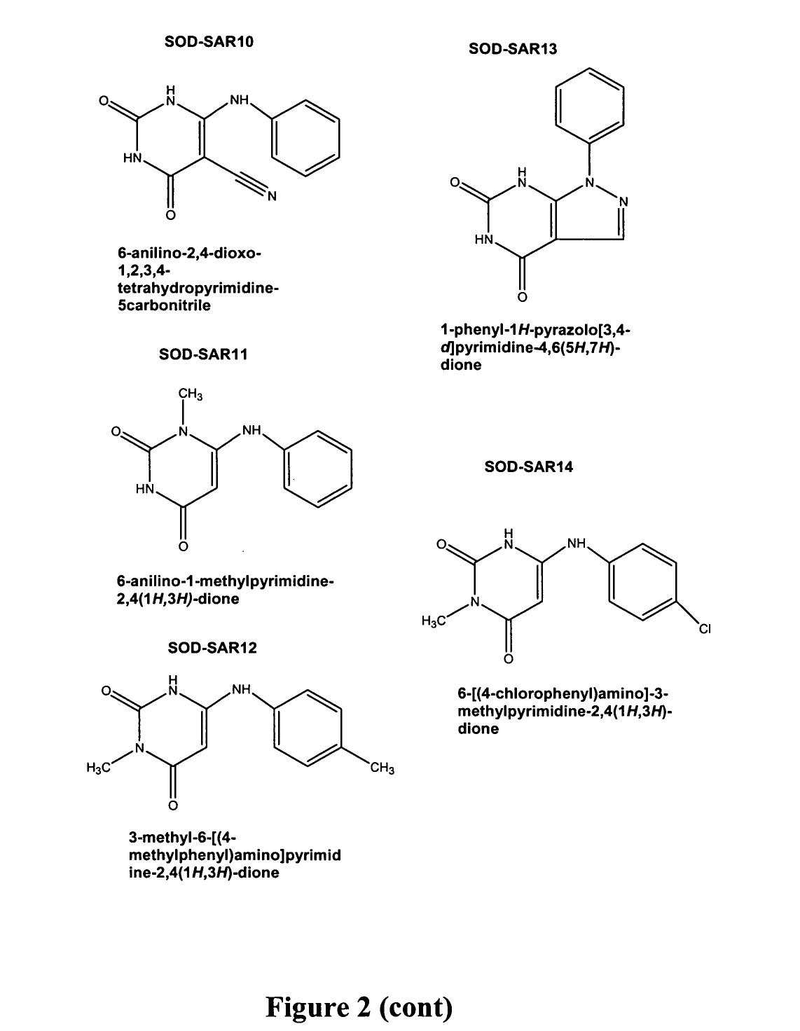

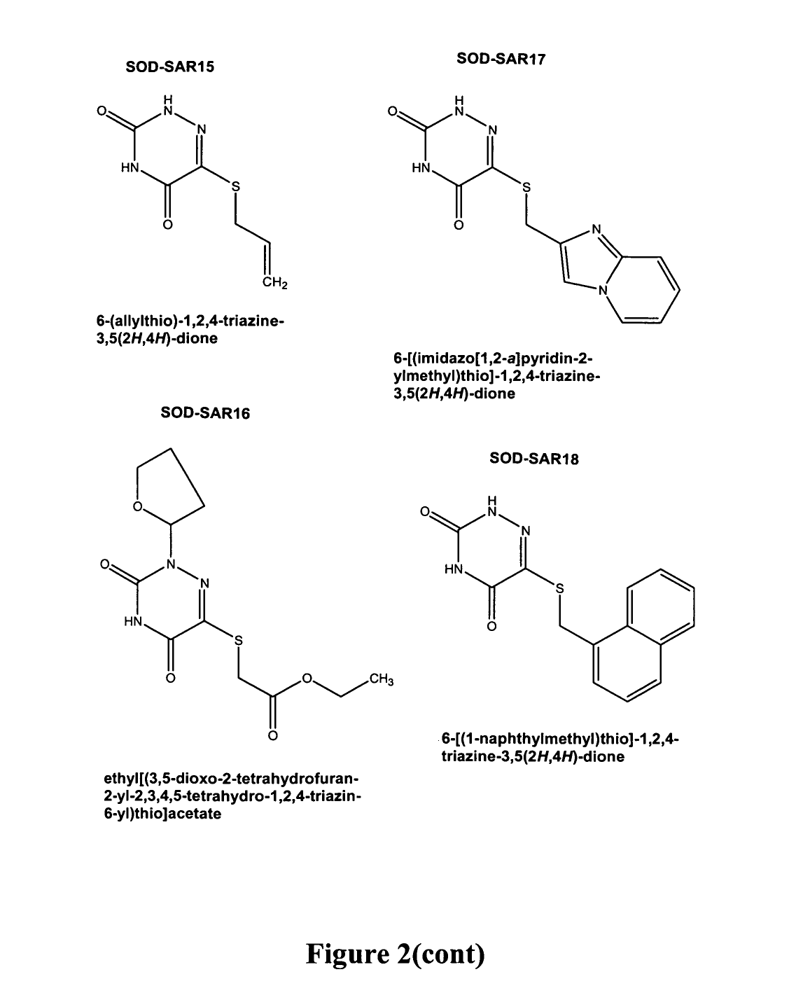

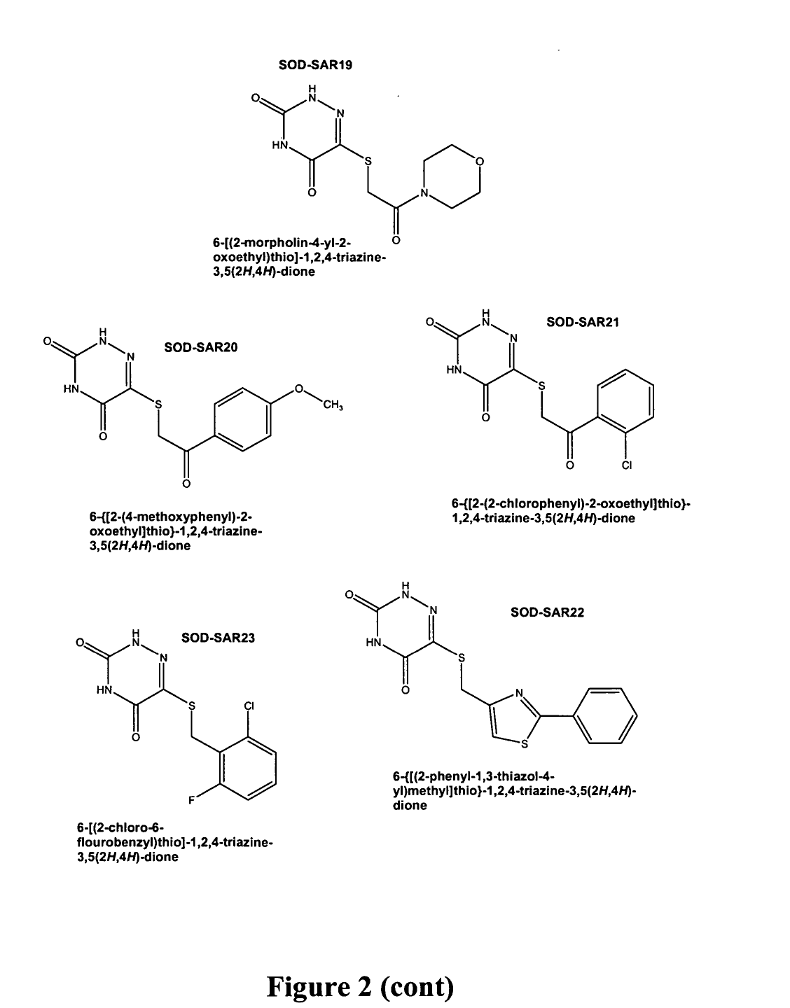

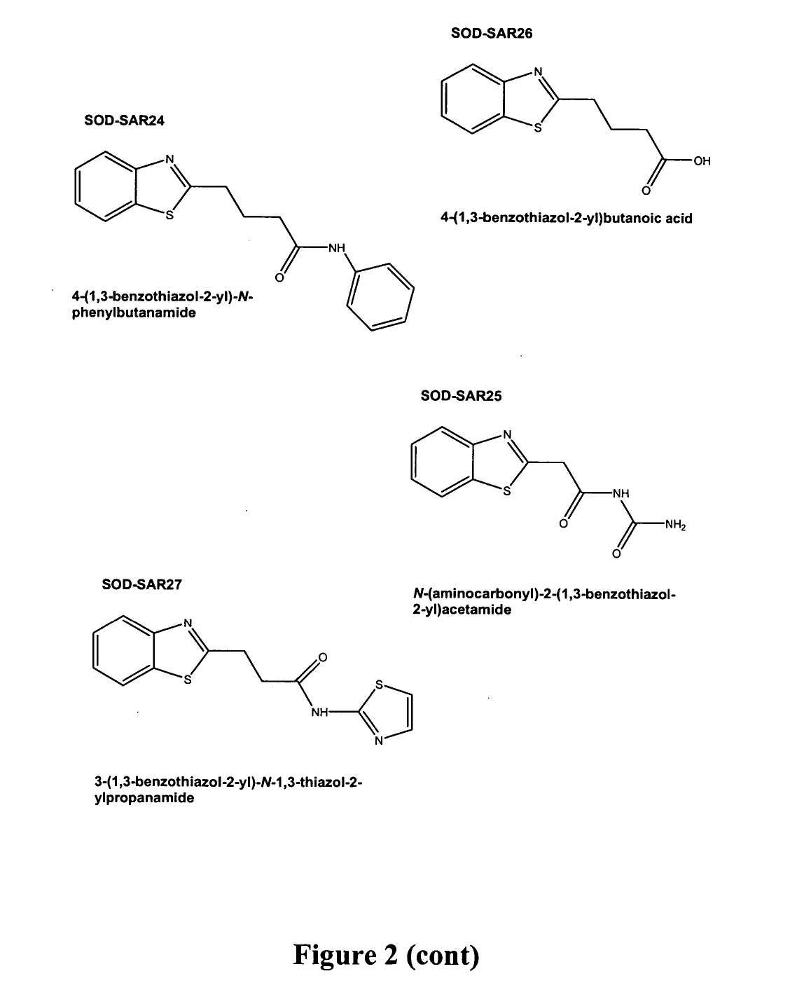

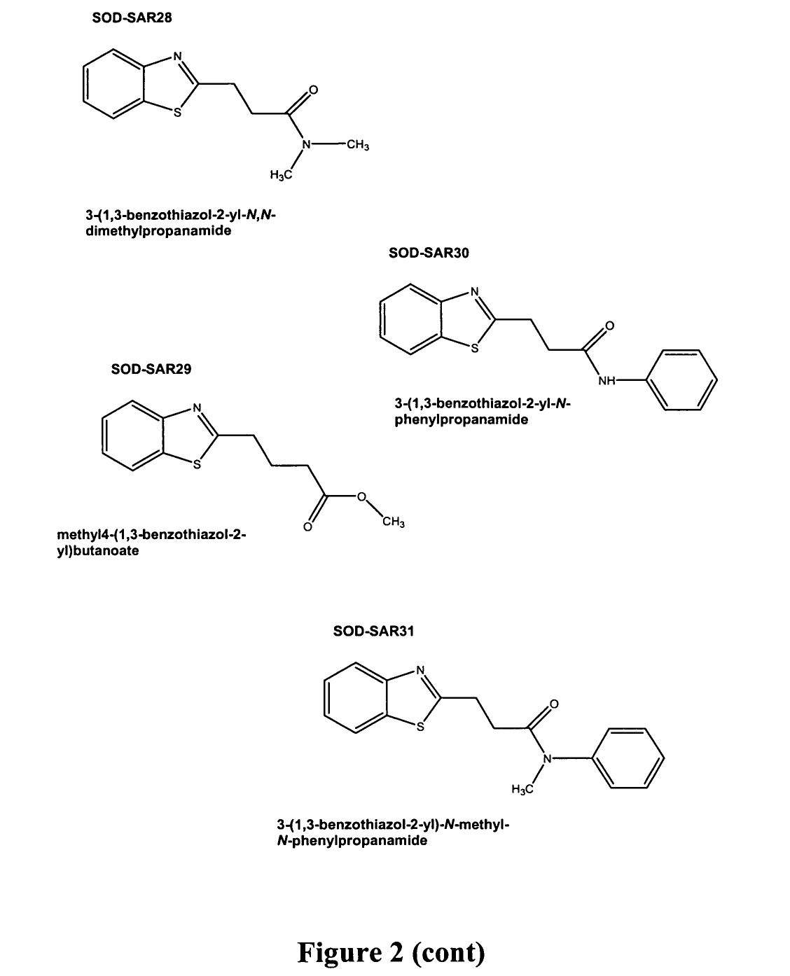

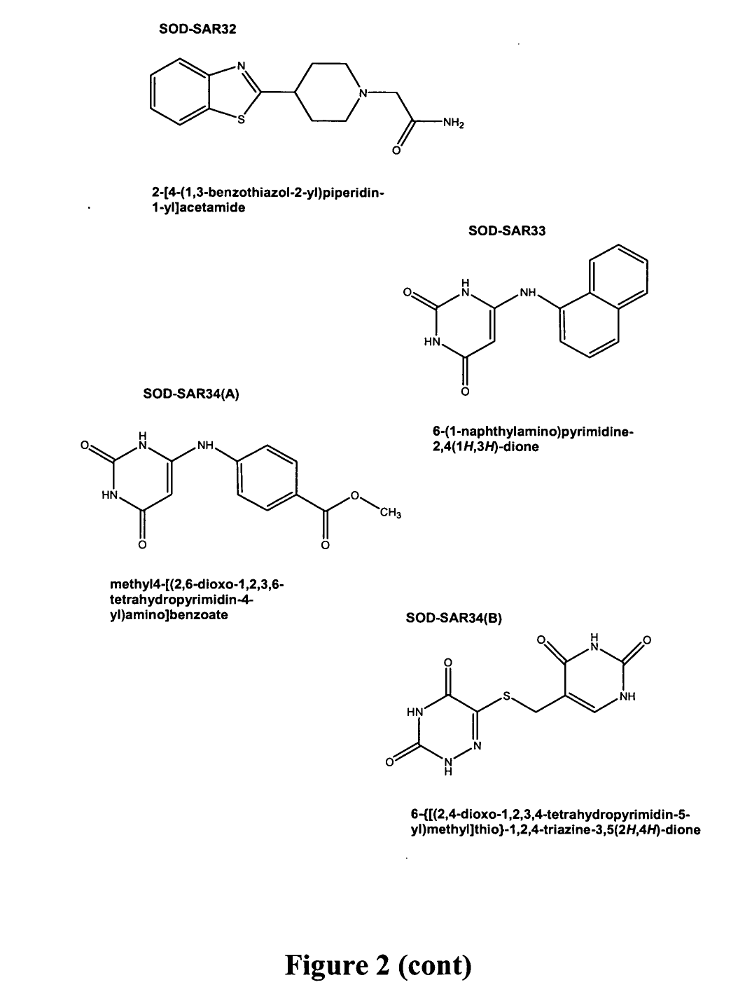

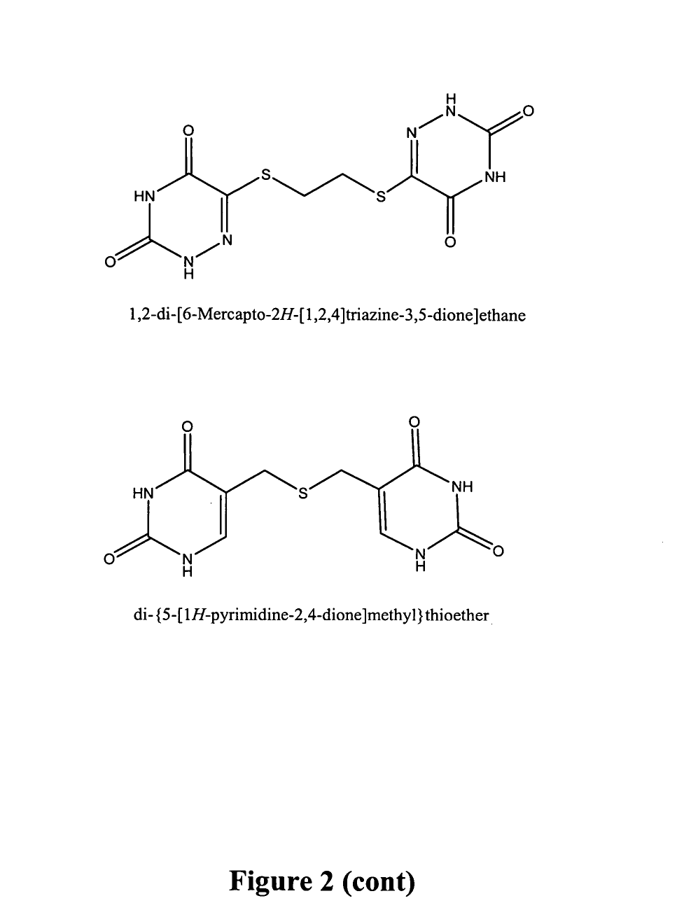

A structural analysis was performed on the 15 compounds found to be active in the assays described in Example 1 (see FIG. 1). Based upon this analysis, a set of structural analogs were identified and are shown in FIG. 2. All of these were tested and found to be effective inhibitors of SOD aggregation with the most active compounds being: 6-{[(2,4-dioxo-1,2,3,4-tetrahydropyrimidin-5-yl)methyl]thio}-1,2,4-triazine-3,5(2H,4H)-dione; 1,2-di-[6-Mercapto-2H-[1,2,4]triazine-3,5-dione]ethane; and di-{5-[1H-pyrimidine-2,4-dione]methyl}thioether. Although most of the compounds tested were purchased commercially, two (1,2-di-[6-Mercapto-2H-[1,2,4]triazine-3,5-dione]ethane; and di-{5-[1H-pyrimidine-2,4-dione]methyl}thioether) were synthesized as described in Example 3.

### Example 3

### Synthesis of Compounds

FIG. 3 shows the reaction scheme that was used in synthesizing two compounds: (1,2-di-[6-Mercapto-2H-[1,2,4]triazine-3,5-dione]ethane; and di-{5-[1H-pyrimidine-2,4-dione]methyl}thioether). Also shown in the figure is a scheme that could, theoretically, be used to synthesize 6-(2,4-Dioxo-1,2,3,4-tetrahydro-pyrimidin-5-ylmethylsulfanyl)-2H-[1,2,4]triazine-3,5-dione. The steps involved are as follows:

A) 5-Bromo-6-azauracil, 5-Mercapto-6-azauracil and 5-Mercaptomethyluracil

5-Bromo-6-azauracil (2) was prepared from 6-azauracil(1) by bromination following the procedure described in the Journal of Organic Chemistry 26:1118-1120 (1961)). 5-Mercapto-6-azauracil (3) was prepared by the procedure described in Die Pharmazie 18:339 (1963)). 5-Mercaptomethyluracil (5) was prepared according to the procedure described in the Journal of Medicinal Chemistry 9:97-101 (1966)).

B) 6-(2,4-Dioxo-1, 2, 3, 4-tetrahydro-pyrimidin-5-ylmethylsulfanyl)-2H-[1,2,4]triazine-3,5-dione

A mixture of 3 (95 mg, 0.5 mmole) and 4 (80 mg, 0.5 mmole) in ethyl alcohol (10 ml) and water (5 ml) is stirred overnight at room temperature. The solid is isolated by filtration, is washed with water (5 ml) and is then dried.

C) 1,2-Di-[6-Mercapto-2H-[1,2,4]triazine-3,5-dione]ethane

A solution of 3 (95 mg, 0.5 mmole) in water (5 ml) was treated dropwise with 1,2-dibromoethane (0.025 m]L, 0.25 mmole) in ethyl alcohol (5 ml) at room temperature and then stirred overnight. The solid was isolated by filtration, washed with water (5 ml) and dried.

D) Di-{5-[1H-pyrimidine-2,4-dione]methyl}thioether

A mixture of 5 (104 mg, 0.66 mmole) and 4 (106 mg, 0.66 mmole) in anhydrous DMF (5 ml) was stirred overnight at 100° C. The reaction mixture was allowed to cool to room temperature and then the solid was isolated by filtration, washed sequentially with DMF (5 ml) and diethyl ether (10 ml) and dried.

### REFERENCES

1. Brown, R., Curr. Opin. Neurobiol. 5:841-6 (1995).
2. Brown, R., Cell 80:687-92 (1995).
3. Bruijn, et al., Neuropathol. Appl. Neurobiol. 22:373-87 (1996).
4. Lindberg, et al., Proc. Nat'l Acad. Sci. USA 99:16607-12 (2002).
5. Johnston, et al., Proc. Nat'l Acad. Sci. USA 97:12571-6 (2000).
6. Hayward, et al., J. Biol. Chem. 277:15923-31 (2002).
7. Tiwari, et al., J. Biol. Chem. 278:5984-92 (2003).
8. Hough, et al., Proc. Nat'l Acad. Sci. USA, in press (2004).
9. Lindberg, et al,. Proc. Nat'l Acad. Sci. USA 101:15893-8 (2004).
10. Khare, et al., Proc. Nat'l Acad. Sci. USA 101:15094-9 (2004).
11. Arnesano, J. Biol. Chem. 279:47998-8003 (2004).
12. Furukawa, et al., EMBO J. 23:2872-81 (2004).
13. Doucette, et al., J. Biol. Chem. (2004).
14. Chung, et al., Biochem. Biophys. Res. Commun. 312:873-6 (2003).
15. Ray, et al., Proc. Nat'l Acad. Sci. USA 101:5701-2 (2004).
16. Ray, et al., Biochemistry 43:4899-905 (2004).
17. Colon, et al., Ciba Found. Symp. 199:228-38; discussion 239-42 (1996).
18. Kelly, et al., Adv. Protein Chem. 50:161-81 (1997).
19. Hammarstrom, et al., Science 293:2459-62 (2001).
20. Miroy, et al., Proc. Nat'l Acad. Sci. USA 93:15051-6 (1996).
21. Oza, et al., Bioorg. Med. Chem. Lett. 9:1-6 (1999).
22. Baures, Bioorg. Med. Chem. 7:1339-47 (1999).
23. McCammon, et. al., Structure (Camb) 10:851-63 (2002).
24. Baures, et. al., Bioorg. Med. Chem. 6:1389-401 (1998).
25. Adamski-Wemer, et. al. J. Med. Chem. 47:355-74 (2004)
26. Halgren, J. Med. Chem. 47:1750-9 (2004).
27. McCord, et al., J. Biol. Chem. 244:6049-55 (1969).
28. Conway, et al., Nat. Med. 4:1318-20 (1998).
29. Kleywegt, et al., Acta Crystallogr. D. Biol. Crystallogr. 55(pt 4):941-4 (1999).
30. Wallace, et al., Biochim. Biophys. Acta 1478:325-32 (2000).
31. Eilers, et al, Proc. Nat'l Acad. Sci. USA 97:5796-801 (2000).
32. Karpusas, et al., Proc. Nat'l Acad. Sci. USA 86:8237-41 (1989).
33. Lipinski, et al., Adv. Drug Deliv. Rev. 46:3-26 (2001).
34. Kontoyianni, et al., J. Med. Chem. 47:558-65 (2004).
35. Santoro, et al., Biochemistry 31:4901-7 (2004).

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of inhibiting the aggregation of superoxide dismutase, comprising contacting superoxide dismutase dimers with an effective amount of a compound of formula I: 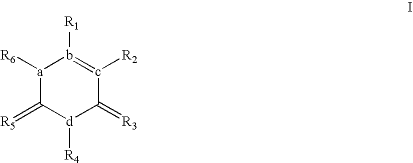
wherein:
a, b, c and d are each independently selected from C and N;
at least one of R₁, R₂ and R₆ is —(CH₂)ₙ-Z-(CH₂)ₘ—R₇,
where Z is C, N or S,
R₇ is H, CH₃ or a phenyl optionally substituted at one or more positions with a halogen, a (C₁-C₃)alkyl, OH or NH₂,
n is an integer from 0 to 3 inclusive,
m is an integer from 0 to 3 inclusive,
and where one or more single bonds in —(CH₂)ₙ-Z-(CH₂)ₘ may be replaced with a double bond;
when R₁ is —(CH₂)ₙ-Z-(CH₂)ₘ—R₇, R₂ and R₆ may also each independently be selected from: H, a halogen, a (C₁-C₃)alkyl, OH and NH₂;
when R₂ is —(CH₂)ₙ-Z-(CH₂)ₘ—R₇, R₁ and R₆ may also each independently be selected from: H, a halogen, a (C₁-C₃)alkyl, OH and NH₂;
when R₆ is —(CH₂)ₙ-Z-(CH₂)ₘ—R₇, R₁ and R₂ may also each independently be selected from: H, a halogen, a (C₁-C₃)alkyl, OH and NH₂;
R₃ and R₅ are each independently either O or S; and
R₄ is H, a halogen, a (C₁-C₃)alkyl, OH or NH₂.

2. The method of claim 1, wherein in said compound of formula I:
a, b, c and d are each independently selected from C and N, with the proviso that a, b, c and d are not all N;
at least one of R₁, R₂ and R₆ is —(CH₂)ₙ-Z-(CH₂)ₘ—R₇,
where Z is C, N or S,
R₇ is H, CH₃ or a phenyl optionally substituted at one or more positions with CH₃ halogen, OH or NH₂,
n is an integer from 0 to 3 inclusive,
m is an integer from 0 to 3 inclusive;
when R₁ is —(CH₂)ₙ-Z-(CH₂)ₘ—R₇, R₂ and R₆ may also each independently be selected from: H, a halogen, a (C₁-C₃)alkyl, OH and NH₂;
when R₂ is —(CH₂)ₙ-Z—(CH₂)ₘ—R₇, R₁ and R₆ may also each independently be selected from: H, a halogen, a (C₁-C₃)alkyl, OH and NH₂;
when R₆ is —(CH₂)ₙ-Z-(CH₂)ₘ—R₇, R₁ and R₂ may also each independently be selected from: H, a halogen, a (C₁-C₃)alkyl, OH and NH₂;
R₃ and R₅ are each independently either O or S; and
R₄ is H, a (C₁-C₃)alkyl, OH or NH₂.

3. The method of claim 2, wherein, in said compound of formula I, a, b, c and d are either C or N, with the proviso that no more than two of a, b, c and d may be N.

4. The method of any one of claims 1-3, wherein, in said compound of formula I, R₁ is H, a halogen, a (C₁-C₃)alkyl, OH or NH₂.

5. The method of any one of claims 1-3, wherein, in said compound of formula I, R₂ is H, a halogen, a (C₁-C₃)alkyl, OH or NH₂.

6. The method of any one of claims 1-3, wherein, in said compound of formula I, R₆ is H, a halogen, a (C₁-C₃)alkyl, OH or NH₂.

7. The method of any one of claims 1-3, wherein, in said compound of formula I, R₁ and R₂ are each independently selected from: H, a halogen, a (C₁-C₃)alkyl, OH and NH₂.

8. The method of any one of claims 1-3, wherein, in said compound of formula I, R₁ and R₆ are each independently selected from: H, a halogen, a (C₁-C₃)alkyl, OH and NH₂.

9. The method of any one of claims 1-3, wherein, in said compound of formula I, R₂ and R₆ are each independently selected from: H, a halogen, a (C₁-C₃)alkyl, OH and NH₂.

10. A method of inhibiting the aggregation of superoxide dismutase, comprising contacting superoxide dismutase dimers with an effective amount of a compound of formula II: 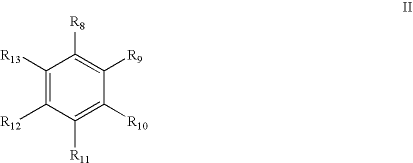
wherein:
R₈, R₉ and R₁₃ are each independently selected from:
group a): 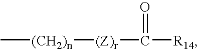
where n is an integer from 0 to 3 inclusive; r is an integer from 1-3 inclusive; Z is C, N or S; and R₁₄ is a (C₁-C₃) alkyl, a halogen, OH, or —NH—R₁₅; where R₁₅ is H, NH₂, OH, or a (C₁-C₃)alkyl;
group b): 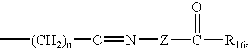
where n is an integer from 0 to 3 inclusive; Z is C, N or S; and R₁₆ is selected from: H, OH, a halogen NH₂, a (C₁-C₃)alkyl, and a phenyl optionally substituted at one or more positions with a halogen, OH, a (C₁-C₃)alkyl, or NH₂;
group c): —(CH₂)ₙ-phenyl, where n is an integer from 0 to 3 inclusive, and the phenyl may optionally be substituted at one or more positions with a halogen, OH, a (C₁-C₃)alkyl or NH₂; and
when R₈ is any of group a), group b), or group c), R₉ and R₁₃ may also each be independently selected from: H, a halogen, a (C₁-C₃)alkyl, OH, and NH₂;
when R₉ is any of group a), group b), or group c), R₈ and R₉ may also each be independently selected from: H, a halogen, a (C₁-C₃)alkyl, OH, and NH₂;
when R₁₃ is any of group a), group b), or group c), R₈ and R₉ may also each be independently selected from: H, a halogen, a (C₁-C₃)alkyl, OH, and NH₂;
R₁₀, R₁₁ and R₁₂ are each independently selected from H, a halogen, a (C₁-C₃)alkyl, OH and NH₂, and any one of R₁₀, R₁₁ and R₁₂ may also be —(CH₂)ₙ-phenyl, wherein n is an integer from 0 to 3 inclusive and the phenyl may optionally be substituted at one or more positions with a halogen, a (C₁-C₃)alkyl, OH or NH₂.

11. The method of claim 10, wherein R₈ is group a), b) or c); and wherein R₉ and R₁₃ are each independently selected from H, a halogen, a (C₁-C₃)alkyl, OH and NH₂.

12. The method of claim 10, wherein R₉ is group a), b) or c); and wherein R₈ and R₁₃ are each independently selected from: H, a halogen, a (C₁-C₃)alkyl, OH and NH₂.

13. The method of claim 10, wherein R₁₃ is group a), b) or c); and wherein R₈ and R₉ are each independently selected from: H, a halogen, a (C₁-C₃)alkyl, OH and NH₂.

14. The method of any one of claims 11-13, wherein R₁₀, R₁₁ and R₁₂ are each independently selected from: a halogen, a (C₁-C₃)alkyl, OH and NH₂.

15. A method of inhibiting the aggregation of superoxide dismutase, comprising contacting superoxide dismutase dimers with an effective amount of a compound of formula III: 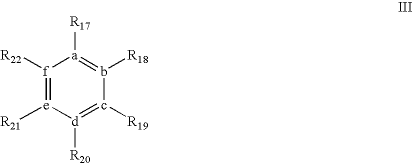
wherein:
a, b, c, d, e, and f are each independently C or N;
at least one of R₁₇, R₁₈ and R₂₂ is 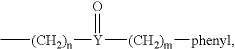
where Y is C or S, n and m are each independently an integer from 0 to 3 inclusive, and the phenyl may optionally be substituted at one or more positions with a halogen, a (C₁-C₃)alkyl, OH or NH₂;
in cases where R₁₇, R₁₈ or R₂₂ is not 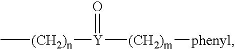
it is independently selected from H, a halogen, a (C₁-C₃)alkyl, OH and NH₂;
at least one of R₁₉, R₂₀ and R₂₁ is 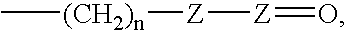
wherein Z is C, N or S, n is an integer from 0 to 3;
in cases where R₁₉, R₂₀ or R₂₁ is not 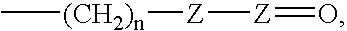
it is independently selected from H, a halogen, a (C₁-C₃)alkyl, OH and NH₂.

16. The method of claim 15, wherein:
a) no more than three of a, b, c, d, e and f are N;
b) no more than one of R₁₇, R₁₈ and R₂₂ is 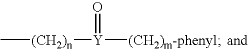
c) no more than one of R₁₉, R₂₀ and R₂₁ is

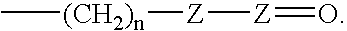

17. The method of claim 16, wherein:
R₁₇ is 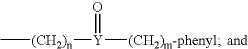
R₂₀ is

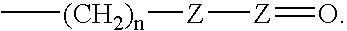

18. The method of any one of claims 15-17, wherein no more than two of a, b, c, d, e and f are N.

19. The method of any one of claims 15-18 wherein no more than one of a, b, c, d, e and f are N.

20. A method of inhibiting the aggregation of superoxide dismutase, comprising contacting superoxide dismutase dimers with an effective amount of a compound of formula IV: 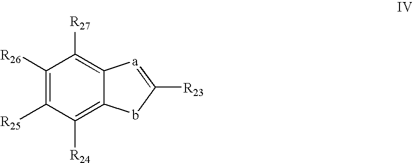
wherein a and b are C, N or S;
R₂₄, R₂₅, R₂₆ and R₂₇ are selected from H, a halogen, a (C₁-C₃)alkyl, OH, and NH₂; and
R₂₃ is 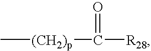
where p is an integer from 0 to 6 inclusive and R₂₈ is selected from a (C₁-C₃)alkyl, OH, a halogen, NH₂, NH—NH₂, and NH—CH₃.

21. The method of claim 20, wherein R₂₈ is NH₂ or NH—NH₂.

22. A method of inhibiting the aggregation of superoxide dismutase, comprising contacting superoxide dismutase dimers with an effective amount of a compound of formula V: 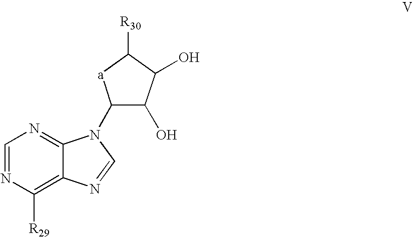
wherein:
a is selected from C, N, or S;
R₂₉ is selected from a halogen, OH, (C₁-C₃)alkyl, and NH₂; and
R₃₀ is —(CH₂)_{q}—PO₄, wherein q is an integer from 1 to 3 inclusive.

23. A method of inhibiting the aggregation of superoxide dismutase, comprising contacting superoxide dismutase dimers with an effective amount of a compound of formula VI: 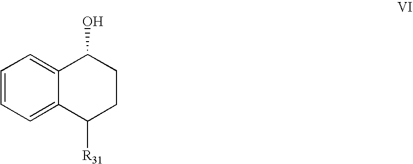
wherein one or more positions in the ring structure of formula VI may be substituted with a group selected from a halogen, a (C₁-C₃)alkyl, OH; and NH₂; and
R₃ is 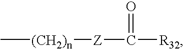
where n is an integer from 0 to 3 inclusive; Z is C, N or S; and R₃₂ is selected from a halogen, OH, a (C₁-C₃)alkyl, and NH₂.

24. The method of claim 23, wherein: n is 1 or 2; Z is N; and R₃₂ is NH₂.

25. A method of inhibiting the aggregation of superoxide dismutase, comprising contacting superoxide dismutase dimers with an effective amount of a compound of formula VII: 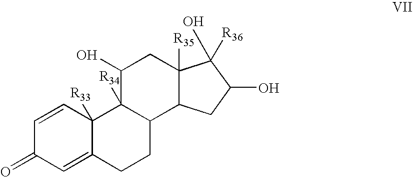
wherein:
R₃₃, R₃₄ and R₃₅ are each independently selected from a halogen, a (C₁-C₃)alkyl, OH and NH₂; and
R₃₆ is 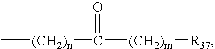
where n and m are each independently an integer from 0 to 3 inclusive and R₃₇ is selected from a halogen, CH₃, OH; and NH₂.

26. A method of inhibiting the aggregation of superoxide dismutase, comprising contacting superoxide dismutase dimers with an effective amount of a compound selected from the group consisting of:
a) N-nitroso-5-(phenylsulfinyl)pyridin-2-amine;
b) 6-[(4-chlorophenyl)amino]pyrimidine-2,4(1H,4H)-dione;
c) 6-[(4-chlorobenzyl)thio]1,2,4-triazine-3,5(2H,4H)-dione;
d) 4-bromo-2-{(E)-[(4-fluorophenyl)imino]methyl}phenol;
e) 6-(ethylthio)-thioxo-4,5-dihydro-1,2,4-triazin-3 (2H)-one;
f) 2-[2-(2-amino-4-methylphenyl)ethyl]-5-methylaniline;
g) 6-[(4-fluorobenzyl)thio]-1,2,4-triazine-3,5(2H,4H)-dione;
h) 2-(3-fluorophenyl)hydrazinecarboxamide;
i) 3-benzyl-2-hydroxylbenzohydrazide;
j) 4-hydroxybenzaldehyde semicarbazone;
k) 4-(1,3-benzothiazol-2-yl)butanamide;
l) 2-(1H-benzimidazol-2-yl)acetohydrazide;
m) N-[(1R,4R)-4-hydroxy-1,2,3,4-tetrahydronaphthalen-1-yl]urea;
n) trimacicolone; and
o) 6-amino-methy-adenosine.

27. A mutated form of SOD having the sequence of SEQ ID NO:1 but wherein at least one amino acid is replaced with a substitute amino acid that can undergo fluorescent labeling, said at least one amino acid being selected from the group consisting of: Gly51; Asp52; Thr54; Aala55; Ser59; Ala1; Thr2; Ala4; Val5; Val7; Lys9; Gly10; Asp11; Gly12; Gln15; Ser107; Gly108; Asp109; Cys111; Ile113; Gly114; Arg115; Thr116; and Leu117.

28. An SOD enzyme having the sequence shown in FIG. 4 wherein said SOD is fluorescently labeled at a position selected from the group consisting of amino acid: 51; 52; 54; 55; 59; 1; 4; 5; 7; 9; 10; 11; 12; 15; 107; 108; 109; 111; 113; 114; 115; 116; and 117; and wherein the amino acid that is shown in each of these positions in FIG. 4 may be replaced with a substitute amino acid to facilitate fluorescent labeling.

29. The method of claim 27, wherein said substitute amino acid is cys.

30. The mutated SOD of claim 27, wherein lysine at position 9 is changed to cys.

31. A dimer of the SOD of any one of claims 27-30.

32. An assay for measuring the ability of a test compound to stabilize SOD dimers, comprising:
a) incubating a labeled SOD homodimer or labeled SOD heterodimer in which both SOD molecules in said homodimer or heterodimer are fluorescently labeled with an unlabeled SOD homodimer in which neither SOD is fluorescently labeled, wherein the incubation is performed in the presence of said test compound;
b) measuring the rate of mixed heterodimer formation in the incubation of step a) by determining the rate of loss of fluorescence attributable to said labeled SOD homodimer or labeled SOD heterodimer over time, wherein said mixed heterodimer has one SOD molecule that is labeled and one SOD molecule that is unlabeled;
c) comparing the rate of mixed heterodimer formation in step b) with the rate determined for a similar incubation carried out in the absence of said test compound; and
d) concluding that said test compound stabilizes the dimerized form of SOD if the rate of mixed heterodimer formation determined in the presence of said test compound is slower than the rate determined in the absence of said test compound.

33. An assay for measuring the ability of a test compound to stabilize SOD dimers, comprising:
a) incubating a first labeled SOD homodimer with a second labeled SOD homodimer in the presence of said test compound, wherein the SOD in said first homodimer is labeled with a donor fluorophore and the SOD in said second homodimer is labeled with an acceptor fluorophore;
b) measuring the rate of mixed donor/acceptor heterodimer formation in the incubation of step a) by measuring the change in fluorescence over time, wherein said mixed donor/acceptor heterodimer has one SOD labeled with said donor fluorophore and one SOD labeled with said acceptor fluorophore;
c) comparing the rate of mixed donor/acceptor heterodimer formation in step b) with the rate determined for a similar incubation carried out in the absence of said test compound; and
d) concluding that said test compound stabilizes the dimerized form of SOD if the rate of mixed donor/acceptor heterodimer formation determined in the presence of said test compound is slower than the rate determined in the absence of said test compound.

34. An assay for measuring the ability of a test compound to stabilize SOD dimers, comprising:
a) incubating a labeled SOD heterodimer with an unlabeled SOD homodimer in the presence of said test compound, wherein said labeled SOD heterodimer has one SOD molecule labeled with a donor fluorophore and a second SOD molecule labeled with an acceptor fluorophore and wherein neither SOD in said SOD homodimer is fluorescently labeled;
b) determining the rate of dimer dissociation by measuring the loss in fluorescence attributable to said labeled SOD heterodimer over time,
c) comparing the rate of fluorescence loss in step b) with the rate determined for a similar incubation carried out in the absence of said test compound; and
d) concluding that said test compound stabilizes the dimerized form of SOD if the rate of fluorescence loss in the presence of said test compound is slower than the rate of fluorescence loss in the absence of said test compound.

35. A method of inhibiting the aggregation of superoxide dismutase, comprising contacting superoxide dismutase dimers with an effective amount of a compound of formula VIII: 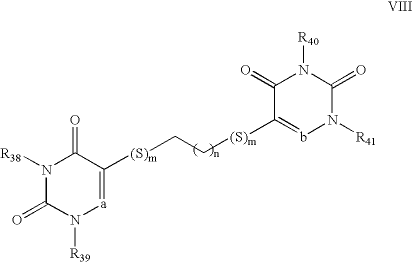
wherein:
R₃₈, R₃₉, R₄₀ and R₄₁ is each independently selected from: H, an alkyl, an aryl, a heteroalkyl, a heteroaryl, or an arylalkyl,
a and b are each CH or N,
n is an integer from 0-6 inclusive,
and m is an integer from 0-1 inclusive.

36. A method of inhibiting the aggregation of superoxide dismutase, comprising contacting superoxide dismutase dimers with an effective amount of a compound of formula IX: 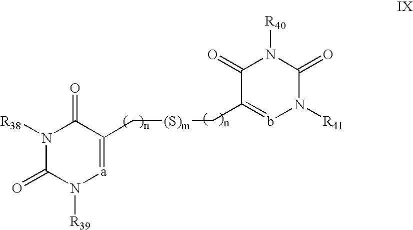
wherein:
R₃₈, R₃₉, R₄₀ and R₄₁ is each independently selected from the group consisting of H, C₁-C₆ alkyl;
a and b are each independently —CH— or —N—;
n is an integer from 0-6 inclusive; and
m is an integer from 0-1 inclusive.

37. A method of inhibiting the aggregation of superoxide dismutase, comprising contacting superoxide dismutase dimers with an effective amount of a compound selected from the group consisting of:
a) methyl3-(3,5-dioxo-2,3,4,5-tetrahydro-1,2,4-triazin-6-yl)propanoate;
b) 6-[(3,5-dimethyl-1H-pyrazol-4-yl)thio]-1,2,4-triazine-3,5(2H,4H)-dione;
c) 3-methyl-6-[methyl(phenyl)amino]pyrimidine-2,4(1H,3H)-dione;
d) 2-(2-methyl-1H-benzimidazol-1-yl)acetamide;
e) hydroxy(oxo) {4-[(2-oxo-1,2,3,6-tetrahydopyrimidin-4-yl)amino]phenyl}ammonium;
f) 6-[(2-chlorophenyl)amino]pyrimidine-2,4(1H,3H)-dione;
g) 6-[(4-pyrrolidin-1-ylphenyl)amino]pyrimidine-2,4(1H,3H)-dione;
h) 6-[(4-methylphenyl)amino]pyrimidine-2,4(1H,3H)-dione;
i) 5-ethylpyrimidine-2,4(1H,3H)-dione;
j) 6-anilino-2,4-dioxo-1,2,3,4-tetrahydropyrimidine-5carbonitrile;
k) 6-anilino-1-methylpyrimidine-2,4(1H,3H)-dione;
l) 3-methyl-6-[(4-methylphenyl)amino]pyrimidine-2,4(1H,3H)-dione;
m) 1-phenyl-1H-pyrazolo[3,4-d]pyrimidine-4,6(5H,7H)-dione;
n) 6-[(4-chlorophenyl)amino]-3-methylpyrimidine-2,4(1H,3H)-dione;
o) 6-(allylthio)-1,2,4-triazine-3,5(2H,4H)-dione;
p) ethyl[(3,5-dioxo-2-tetrahydrofuran-2-yl-2,3,4,5-tetrahydro-1,2,4-triazin-6-yl)thio]acetate;
q) 6-[(imidazo[1,2-a]pyridin-2-ylmethyl)thio]-1,2,4-triazine-3,5(2H,4H)-dione;
r) 6-[(1-naphthylmethyl)thio]-1,2,4-triazine-3,5(2H,4H)-dione;
s) 6-[(2-morpholin-4-yl-2-oxoethyl)thio]-1,2,4-triazine-3,5(2H,4H)-dione;
t) 6-{[2-(4-methoxyphenyl)-2-oxoethyl]thio}-1,2,4-triazine-3,5(2H,4H)-dione;
u) 6-{[2-(2-chlorophenyl)-2-oxoethyl]thio}-1,2,4-triazine-3,5(2H,4H)-dione;
v) 6-{[(2-phenyl-1,3-thiazol-4-yl)methyl]thio}-1,2,4-triazine-3,5(2H,4H)-dione;
w) 6-[(2-chloro-6-flourobenzyl)thio]-1,2,4-triazine-3,5(2H,4H)-dione;
x) 4-(1,3-benzothiazol-2-yl)-N-phenylbutanamide;
y) N-(aminocarbonyl)-2-(1,3-benzothiazol-2-yl)acetamide;
z) 4-(1,3-benzothiazol-2-yl)butanoic acid;
aa) 3-(1,3-benzothiazol-2-yl)-N-1,3-thiazol-2-ylpropanamide;
bb) 3-(1,3-benzothiazol-2-yl-NN-dimethylpropanamide;
cc) methyl-4-(1,3-benzothiazol-2-yl)butanoate;
dd) 3-(1,3-benzothiazol-2-yl-N-phenylpropanamide;
ee) 3-(1,3-benzothiazol-2-yl)-N-methyl-N-phenylpropanamide;
ff) 2-[4-(1,3-benzothiazol-2-yl)piperidin-1-yl]acetamide;
gg) 6-(1-naphthylamino)pyrimidine-2,4(1H,3H)-dione;
hh) methyl-4-[(2,6-dioxo-1,2,3,6-tetrahydropyrimidin-4-yl)amino]benzoate;
ii) 6-{[(2,4-dioxo-1,2,3,4-tetrahydropyrimidin-5-yl)methyl]thio}-1,2,4-triazine-3,5(2H,4H)-dione;
jj) 1,2-di-[6-Mercapto-2H-[1,2,4]triazine-3,5-dione]ethane; and
kk) di-{5-[1H-pyrimidine-2,4-dione]methyl}thioether.

38. A compound selected from the group consisting of:
1,2-di-[6-Mercapto-2H-[1,2,4]triazine-3,5-dione]ethane; and
di-{5-[1H-pyrimidine-2,4-dione]methyl}thioether.

39. A pharmaceutical composition in unit dosage form comprising a therapeutically effective amount of a compound selected from the group consisting of:
a) N-nitroso-5-(phenylsulfinyl)pyridin-2-amine;
b) 6-[(4-chlorophenyl)amino]pyrimidine-2,4(1H,4H)-dione;
c) 6-[(4-chlorobenzyl)thio]1,2,4-triazine-3,5(2H,4H)-dione;
d) 4-bromo-2-{(E)-[(4-fluorophenyl)imino]methyl}phenol;
e) 6-(ethylthio)-thioxo-4,5-dihydro-1,2,4-triazin-3(2H)-one;
f) 2-[2-(2-amino-4-methylphenyl)ethyl]-5-methylaniline;
g) 6-[(4-fluorobenzyl)thio]-1,2,4-triazine-3,5(2H,4H)-dione;
h) 2-(3-fluorophenyl)hydrazinecarboxamide;
i) 3-benzyl-2-hydroxylbenzohydrazide;
j) 4-hydroxybenzaldehyde semicarbazone;
k) 4-(1,3-benzothiazol-2-yl)butanamide;
l) 2-(1H-benzimidazol-2-yl)acetohydrazide;
m) N-[(1R,4R)-4-hydroxy-1,2,3,4-tetrahydronaphthalen-1-yl]urea;
n) trimacicolone;
o) 6-amino-methy-adenosine;
p) methyl3-(3,5-dioxo-2,3,4,5-tetrahydro-1,2,4-triazin-6-yl)propanoate;
q) 6-[(3,5-dimethyl-1H-pyrazol-4-yl)thio]-1,2,4-triazine-3,5(2H,4H)-dione;
r) 3-methyl-6-[methyl(phenyl)amino]pyrimidine-2,4(1H,3H)-dione;
s) 2-(2-methyl-1H-benzimidazol-1-yl)acetamide;
t) hydroxy(oxo) {4-[(2-oxo-1,2,3,6-tetrahydopyrimidin-4-yl)amino]phenyl}ammonium;
u) 6-[(2-chlorophenyl)amino]pyrimidine-2,4(1H,3H)-dione;
v) 6-[(4-pyrrolidin-1-ylphenyl)amino]pyrimidine-2,4(1H,3H)-dione;
w) 6-[(4-methylphenyl)amino]pyrimidine-2,4(1H,3H)-dione;
x) 5-ethylpyrimidine-2,4(1H,3H)-dione;
y) 6-anilino-2,4-dioxo-1,2,3,4-tetrahydropyrimidine-5carbonitrile;
z) 6-anilino-1-methylpyrimidine-2,4(1H,3H)-dione;
aa) 3-methyl-6-[(4-methylphenyl)amino]pyrimidine-2,4(1H,3H)-dione;
bb) 1-phenyl-1H-pyrazolo[3,4-d]pyrimidine-4,6(5H,7H)-dione;
cc) 6-[(4-chlorophenyl)amino]-3-methylpyrimidine-2,4(1H,3H)-dione;
dd) 6-(allylthio)-1,2,4-triazine-3,5(2H,4H)-dione;
ee) ethyl[(3,5-dioxo-2-tetrahydrofuran-2-yl-2,3,4,5-tetrahydro-1,2,4-triazin-6-yl)thio]acetate;
ff) 6-[(imidazo[1,2-a]pyridin-2-ylmethyl)thio]-1,2,4-triazine-3,5(2H,4H)-dione;
gg) 6-[(1-naphthylmethyl)thio]-1,2,4-triazine-3,5(2H,4H)-dione;
hh) 6-[(2-morpholin-4-yl-2-oxoethyl)thio]-1,2,4-triazine-3,5(2H,4H)-dione;
ii) 6-{[2-(4-methoxyphenyl)-2-oxoethyl]thio}-1,2,4-triazine-3,5(2H,4H)-dione;
jj) 6-{[2-(2-chlorophenyl)-2-oxoethyl]thio}-1,2,4-triazine-3,5 (2H,4H)-dione;
kk) 6-{[(2-phenyl-1,3-thiazol-4-yl)methyl]thio}-1,2,4-triazine-3,5 (2H,4H)-dione;
ll) 6-[(2-chloro-6-flourobenzyl)thio]-1,2,4-triazine-3,5(2H,4H)-dione;
mm) 4-(1,3-benzothiazol-2-yl)-N-phenylbutanamide;
nn) N-(aminocarbonyl)-2-(1,3-benzothiazol-2-yl)acetamide;
oo) 4-(1,3-benzothiazol-2-yl)butanoic acid;
pp) 3-(1,3-benzothiazol-2-yl)-N-1,3-thiazol-2-ylpropanamide;
qq) 3-(1,3-benzothiazol-2-yl-N,N-dimethylpropanamide;
rr) methyl-4-(1,3-benzothiazol-2-yl)butanoate;
ss) 3-(1,3-benzothiazol-2-yl-N-phenylpropanamide;
tt) 3-(1,3-benzothiazol-2-yl)-N-methyl-N-phenylpropanamide;
uu) 2-[4-(1,3-benzothiazol-2-yl)piperidin-1-yl]acetamide;
vv) 6-(1-naphthylamino)pyrimidine-2,4(1H,3H)-dione;
ww) methyl-4-[(2,6-dioxo-1,2,3,6-tetrahydropyrimidin-4-yl)amino]benzoate;
xx) 6-{[(2,4-dioxo-1,2,3,4-tetrahydropyrimidin-5-yl)methyl]thio}-1,2,4-triazine-3,5(2H,4H)-dione;
yy) 1,2-di-[6-Mercapto-2H-[1,2,4]triazine-3,5-dione]ethane;
zz) di-{5-[1H-pyrimidine-2,4-dione]methyl}thioether.


SEQUENCE LISTING (6 sequences)

SEQ ID NO: 1 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 2 - DNA, 459 bp, Homo sapiens
gcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaa

SEQ ID NO: 3 - protein, 153 aa, Homo sapiens
ATKAVCVLCGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 4 - DNA, 459 bp, Homo sapiens
gcgacgaaggccgtgtgcgtgctgtgtggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaa

SEQ ID NO: 5 - protein, 153 aa, Homo sapiens
ATKVVCVLCGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 6 - DNA, 459 bp, Homo sapiens
gcgacgaaggtcgtgtgcgtgctgtgtggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaa